(19) United States
(12) Patent Application Publication
(10) Pub. No.: US 2011/0112147 A1
(43) Pub. Date: May 12, 2011

(54) INDAZOLONE ANALOGS AS GLYCOGEN SYNTHASE ACTIVATORS

(76) Inventors: David Robert Bolin, Montclair, NJ (US); Stuart Hayden, Manalapan, NJ (US); Yimin Qian, Wayne, NJ (US)

(21) Appl. No.: 12/897,860

(22) Filed: Oct. 5, 2010

Related U.S. Application Data
(60) Provisional application No. 61/260,055, filed on Nov. 11, 2009.

(51) Int. Cl. A61K 31/4439; C07D 231/56; A61K 31/416; C07D 401/04; A61P 3/00

(52) U.S. Cl. 514/338; 548/361.5; 514/405; 546/275.7

(57) ABSTRACT

Provided herein are compounds of the formula (I):

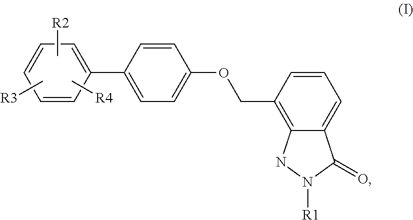

as well as pharmaceutically acceptable salts thereof, wherein the substituents are as those disclosed in the specification. These compounds, and the pharmaceutical compositions containing them, are useful for the treatment of metabolic diseases and disorders such as, for example, type II diabetes mellitus.

### PRIORITY TO RELATED APPLICATION(S)

This application claims the benefit of U.S. Provisional Application No. 61/260,055, filed Nov. 11, 2009, which is hereby incorporated by reference in its entirety.

### FIELD OF THE INVENTION

The invention is directed to compounds, salts and pharmaceutical compositions useful as activators of glycogen synthase for the treatment of metabolic diseases and disorders.

All documents cited or relied upon below are expressly incorporated herein by reference.

### BACKGROUND OF THE INVENTION

Diabetes mellitus is a common and serious disorder, affecting 10 million people in the U.S. [Harris, M. I. Diabetes Care 1998 21 (3S) Supplement, 11C], putting them at increased risk of stroke, heart disease, kidney damage, blindness, and amputation. Diabetes is characterized by decreased insulin secretion and/or an impaired ability of peripheral tissues to respond to insulin, resulting in increased plasma glucose levels. The incidence of diabetes is increasing, and the increase has been associated with increasing obesity and a sedentary life. There are two forms of diabetes: insulin-dependent and non-insulin-dependent, with the great majority of diabetics suffering from the non-insulin-dependent form of the disease, known as type 2 diabetes or non-insulin-dependent diabetes mellitus (NIDDM). Because of the serious consequences, there is an urgent need to control diabetes.

Treatment of NIDDM generally starts with weight loss, a healthy diet and an exercise program. However, these factors are often unable to control the disease, and there are a number of drug treatments available, including insulin, metformin, sulfonylureas, acarbose, and thiazolidinediones. Each of these treatments has disadvantages and there is an ongoing need for new drugs to treat diabetes.

Metformin is an effective agent that reduces fasting plasma glucose levels and enhances the insulin sensitivity of peripheral tissue, mainly through an increase in glycogen synthesis [De Fronzo, R. A. Drugs 1999, 58 Suppl. 1, 29]. Metformin also leads to reductions in the levels of LDL cholesterol and triglycerides [Inzucchi, S. E. JAMA 2002, 287, 360]. However, it loses its effectiveness over a period of years [Turner, R. C. et al. JAMA 1999, 281, 2005].

Thiazolidinediones are activators of the nuclear receptor peroxisome-proliferator activated receptor-gamma. They are effective in reducing blood glucose levels, and their efficacy has been attributed primarily to decreasing insulin resistance in skeletal muscle [Tadayyon, M. and Smith, S. A. Expert Opin. Investig. Drugs 2003, 12, 307]. One disadvantage associated with the use of thiazolidinediones is weight gain.

Sulfonylureas bind to the sulfonylurea receptor on pancreatic beta cells, stimulate insulin secretion, and consequently reduce blood glucose levels. Weight gain is also associated with the use of sulfonylureas [Inzucchi, S. E. JAMA 2002, 287, 360] and, like metformin, they lose efficacy over time [Turner, R. C. et al. JAMA 1999, 281, 2005]. A further problem often encountered in patients treated with sulfonylureas is hypoglycemia [Salas, M. and Caro, J. J. Adv. Drug React. Tox. Rev. 2002, 21, 205-217].

Acarbose is an inhibitor of the enzyme alpha-glucosidase, which breaks down disaccharides and complex carbohydrates in the intestine. It has lower efficacy than metformin or the sulfonylureas, and it causes intestinal discomfort and diarrhea which often lead to the discontinuation of its use [Inzucchi, S. E. JAMA 2002, 287, 360].

Because none of these treatments is effective over the long term without serious side effects, there is a need for new drugs for the treatment of type 2 diabetes.

In skeletal muscle and liver, there are two major pathways of glucose utilization: glycolysis, or oxidative metabolism, where glucose is oxidized to pyruvate; and glycogenesis, or glucose storage, where glucose is stored in the polymeric form glycogen. The key step in the synthesis of glycogen is the addition of the glucose derivative UDP-glucose to the growing glycogen chain, and this step is catalyzed by the enzyme glycogen synthase [Cid, E. et al. J. Biol. Chem. 2000, 275, 33614]. There are two isoforms of glycogen synthase, found in liver [Bai, G. et al. J. Biol. Chem. 1990, 265, 7843] and in other peripheral tissues including muscle [Browner, M. F. et al. Proc. Nat. Acad. Sci. U.S.A. 1989, 86, 1443]. There is clinical and genetic evidence implicating both forms of glycogen synthase in metabolic diseases such as type 2 diabetes and cardiovascular disease. Both basal and insulin-stimulated glycogen synthase activity in muscle cells from diabetic subjects were significantly lower than in cells from lean non-diabetic subjects [Henry, R. R. et al. J. Clin. Invest. 1996, 98, 1231-1236; Nikoulina, S. E. et al. J. Clin. Enocrinol. Metab. 2001, 86, 4307-4314]. Furthermore, several studies have shown that levels of muscle [Eriksson, J. et al. N. Engl. J. Mod. 1989, 331, 337; Schulman, R. G. et al. N. Engl. J. Med. 1990, 332, 223; Thorburn, A. W. et al. J. Clin. Invest. 1991, 87, 489] and liver [Krssak, M. et. al. Diabetes 2004, 53, 3048] glycogen are lower in diabetic patients than in control subjects. In addition, genetic studies have shown associations in several populations between type 2 diabetes and/or cardiovascular disease and mutation/deletion in the GYS1 gene encoding the muscle isoform of glycogen synthase [Orhu-Melander, M. et al. Diabetes 1999, 48, 918; Fredriksson, J. et. al. PLoS ONE 2007, 3, e285; Kolhberg G. et. al. N. Engl. J. Med. 2007, 357, 1507]. Patients lacking GYS2 encoding the liver isoform of glycogen synthase, suffer from fasting ketotic hypoglycemia and postprandial hyperglycemia, hyperlactanemia and hyperlipidemia, supporting the essential role of liver GS in maintaining normal nutrient metabolism. [Weinstein, D. A. et. al. Mol. Genetics and Metabolism, 2006, 87, 284]

Glycogen synthase is subject to complex regulation, involving phosphorylation in at least nine sites [Lawrence, J. C., Jr. and Roach, P. J. Diabetes 1997, 46, 541]. The dephosphorylated form of the enzyme is active. Glycogen synthase is phosphorylated by a number of enzymes of which glycogen synthase kinase 3β (GSK3β) is the best understood [Tadayyon, M. and Smith, S. A. Expert Opin. Investig. Drugs 2003, 12, 307], and glycogen synthase is dephosphorylated by protein phosphatase type I (PP1) and protein phosphatase type 2A (PP2A). In addition, glycogen synthase is regulated by an endogenous ligand, glucose-6-phosphate which allosterically stimulates the activity of glycogen synthase by causing a change in the conformation of the enzyme that renders it more susceptible to dephosphorylation by the protein phosphatases to the active form of the enzyme [Gomis, R. R. et al. J. Biol. Chem. 2002, 277, 23246].

Several mechanisms have been proposed for the effect of insulin in reducing blood glucose levels, each resulting in an increase in the storage of glucose as glycogen. First, glucose uptake is increased through recruitment of the glucose transporter GLUT4 to the plasma membrane [Holman, G. D. and Kasuga, M. Diabetologia 1997, 40, 991]. Second, there is an increase in the concentration of glucose-6-phosphate, the allosteric activator of glycogen synthase [Villar-Palasi, C. and Guinovart, J. J. FASEB J. 1997, 11, 544]. Third, a kinase cascade beginning with the tyrosine kinase activity of the insulin receptor results in the phosphorylation and inactivation of GSK3β, thereby preventing the deactivation of glycogen synthase [Cohen, P. Biochem. Soc. Trans. 1993, 21, 555; Yeaman, S. J. Biochem. Soc. Trans. 2001, 29, 537].

Because a significant decrease in the activity of glycogen synthase has been found in diabetic patients, and because of its key role in glucose utilization, the activation of the enzyme glycogen synthase holds therapeutic promise for the treatment of metabolic diseases such as type 2 diabetes and cardiovascular diseases. The only known allosteric activators of the enzyme are glucose-6-phosphate [Leloir, L. F. et al. Arch. Biochem. Biophys. 1959, 81, 508] and glucosamine-6-phosphate [Virkamaki, A. and Yki-Jarvinen, H. Diabetes 1999, 48, 1101].

The following biaryloxymethylarenecarboxylic acids are reported to be commercially available from Otava, Toronto, Canada, Akos Consulting & Solutions, Steinen, Germany or Princeton BioMolecular Research, Monmouth Junction, N.J.: 4-(biphenyl-4-yloxymethyl)-benzoic acid, 3-(biphenyl-4-yloxymethyl)-benzoic acid, [4-(biphenyl-4-yloxymethyl)-phenyl]-acetic acid, [4-(4′-methyl-biphenyl-4-yloxymethyl)-phenyl]-acetic acid, 4-(4′-methyl-biphenyl-4-yloxymethyl)-benzoic acid, 3-(3-bromo-biphenyl-4-yloxymethyl)-benzoic acid, [4-(3-bromo-biphenyl-4-yloxymethyl)-phenyl]-acetic acid, 2-(4′-methyl-biphenyl-4-yloxymethyl)-benzoic acid, 5-(biphenyl-4-yloxymethyl)-furan-2-carboxylic acid, 5-(4′-methyl-biphenyl-4-yloxymethyl)-furan-2-carboxylic acid, 5-(3-bromo-biphenyl-4-yloxymethyl)-furan-2-carboxylic acid, 4-(biphenyl-4-yloxymethyl)-5-methyl-furan-2-carboxylic acid, 5-methyl-4-(4′-methyl-biphenyl-4-yloxymethyl)-furan-2-carboxylic acid, 4-(3-bromo-biphenyl-4-yloxymethyl)-5-methyl-furan-2-carboxylic acid, 2-(biphenyl-4-yloxymethyl)-4-methyl-thiazole-5-carboxylic acid, [2-(biphenyl-4-yloxymethyl)-thiazol-4-yl]-acetic acid, [2-(4′-methyl-biphenyl-4-yloxymethyl)-thiazol-4-yl]-acetic acid and [5-(biphenyl-4-yloxymethyl)-[1,3,4]oxadiazol-2-yl]-acetic acid.

Some biaryloxymethylarenecarboxylic acids are known in the art. However, none of these known compounds have been associated with either the treatment of diseases mediated by the activation of the glycogen synthase enzyme or to any pharmaceutical composition for the treatment of diseases mediated by the activation of the glycogen synthase enzyme. Andersen, H. S. et al. WO 9740017 discloses the structure and synthetic route to 3-(biphenyl-4-yloxymethyl)-benzoic acid as an intermediate in the synthesis of SH2 inhibitors. Winkelmann, E. et al. DE 2842243 discloses 5-(biphenyl-4-yloxymethyl)-thiophene-2-carboxylic acid as a hypolipemic agent. Mueller, T. et al. DE 4142514 discloses 2-(biphenyl-3-yloxymethyl)-benzoic acid as a fungicide. Ghosh, S. S. et al. WO 2004058679 discloses biaryloxymethylarene acids as ligands of adenine nucleoside translocase. Van Zandt, M. C. WO 2008033455 discloses biphenyl and heteroarylphenyl derivatives as protein phosphatase-1B inhibitors.

Glycogen synthase activators and stimulators of glycogen production have been reported. Chu, C. A et al. US 20040266856 discloses biaryoxymethylarenecarboxylic acids as glycogen synthase activators. Chu, C. A. WO 2005000781 discloses biaryloxymethylarene carboxylic acids as activators of glycogen synthase. Yang, S-P. and Huang, Y. US 20050095219 discloses hyaluronic acid compounds that stimulate glycogen production. Gillespie, P. et al. WO 2005075468 discloses biaryoxymethylarene carboxylic acids as glycogen synthase activators. Gillespie, P. et al. WO 2006058648 discloses biaryoxymethylarene carboxylic acids as glycogen synthase activators. Bucala, R. et al. WO 2007044622 discloses macrophage migration inhibitory factor agonists that stimulate glycogen production.

### SUMMARY OF THE INVENTION

The present invention is directed to compounds of the formula I:

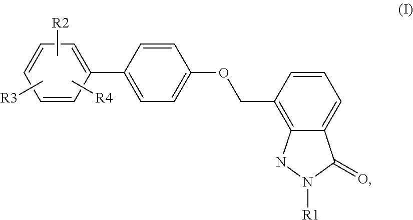

as well as pharmaceutically acceptable salts thereof, pharmaceutical compositions containing them and to methods of treating diseases and disorders. The compounds and compositions disclosed herein are glycogen synthase activators and are useful for the treatment of metabolic diseases and disorders, preferably diabetes mellitus, more preferably type II diabetes mellitus.

### DETAILED DESCRIPTION OF THE INVENTION

In an embodiment of the present invention, provided is a compound of formula (I):

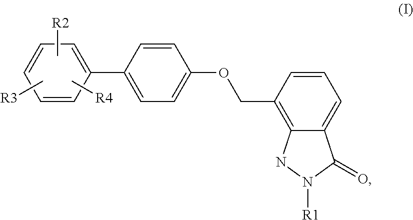

wherein:
R1 is heteroaryl, unsubstituted aryl, aryl substituted with halogen, unsubstituted lower alkyl or lower alkyl mono- or bi-substituted with hydroxy, alkoxy or —COOH; and
R2, R3, R4, independently of each other, is halogen, lower alkyl or alkoxy,
or a pharmaceutically acceptable salt thereof.
   Preferably, R1 is lower alkyl; and R2, R3, R4, independently of each other, is halogen, lower alkyl or alkoxy.

Preferably, R1 is lower alkyl mono- or bi-substituted with hydroxy, alkoxy or —COOH; and R2, R3, R4, independently of each other, is halogen, lower alkyl or alkoxy.

Preferably, R1 is lower alkyl; R2 and R3 are halogen; and R4 is lower alkyl.

Preferably, R2 and R3 are halogen and R4 is lower alkyl.

Preferably, R1 is methyl, acetic acid, methoxy-ethyl, hydroxy-ethyl or dihydroxy-propyl.

Preferably, R1 is phenyl, fluoro-phenyl or pyridinyl.

Preferably, R2 is fluorine or chlorine.

Preferably, R3 is fluorine or chlorine.

Preferably, R4 is chlorine, fluorine, methyl or methoxy.

Preferably, the compound according to formula (I) is:
7-(4′,5′-Difluoro-2′-methyl-biphenyl-4-yloxymethyl)-2-methyl-1,2-dihydro-indazol-3-one;
2-Methyl-7-(2′,4′,5′-trifluoro-biphenyl-4-yloxymethyl)-1,2-dihydro-indazol-3-one;
7-(4′,5′-Difluoro-2′-methoxy-biphenyl-4-yloxymethyl)-2-methyl-1,2-dihydro-indazol-3-one;
[3-oxo-7-(2′,4′,5′-trifluoro-biphenyl-4-yloxymethyl)-1,3-dihydro-indazol-2-yl]-acetic acid;
[7-(4′,5′-Difluoro-2′-methoxy-biphenyl-4-yloxymethyl)-3-oxo-1,3-dihydro-indazol-2-yl]-acetic acid;
2-(2-Methoxy-ethyl)-7-(2′,4′,5′-trifluoro-biphenyl-4-yloxymethyl)-1,2-dihydro-indazol-3-one Hydrochloride;
7-(4′,5′-Difluoro-2′-methoxy-biphenyl-4-yloxymethyl)-2-(2-methoxy-ethyl)-1,2-dihydro-indazol-3-one;
2-(2-Hydroxy-ethyl)-7-(2′,4′,5′-trifluoro-biphenyl-4-yloxymethyl)-1,2-dihydro-indazol-3-one;
[7-(4′,5′-Difluoro-2′-methyl-biphenyl-4-yloxymethyl)-3-oxo-1,3-dihydro-indazol-2-yl]-acetic acid hydrochloride;
7-(4′,5′-Difluoro-2′-methyl-biphenyl-4-yloxymethyl)-2-(4-fluoro-phenyl)-1,2-dihydro-indazol-3-one;
7-(4′,5′-Difluoro-2′-methoxy-biphenyl-4-yloxymethyl)-2-(4-fluoro-phenyl)-1,2-dihydro-indazol-3-one;
7-(4′,5′-Difluoro-2′-methoxy-biphenyl-4-yloxymethyl)-2-pyridin-3-yl-1,2-dihydro-indazol-3-one;
2-Pyridin-3-yl-7-(2′,4′,5′-trifluoro-biphenyl-4-yloxymethyl)-1,2-dihydro-indazol-3-one Hydrochloride;
7-(4′,5′-Difluoro-2′-chloro-biphenyl-4-yloxymethyl)-2-pyridin-3-yl-1,2-dihydro-indazol-3-one;
2-((R)-2,3-Dihydroxy-propyl)-7-(2′,4′,5′-trifluoro-biphenyl-4-yloxymethyl)-1,2-dihydro-indazol-3-one;
2-((S)-2,3-Dihydroxy-propyl)-7-(2′,4′,5′-trifluoro-biphenyl-4-yloxymethyl)-1,2-dihydro-indazol-3-one;
7-(4′,5′-Difluoro-2′-methoxy-biphenyl-4-yloxymethyl)-2-((R)-2,3-dihydroxy-propyl)-1,2-dihydro-indazol-3-one;
7-(4′,5′-Difluoro-2′-methoxy-biphenyl-4-yloxymethyl)-2-((S)-2,3-dihydroxy-propyl)-1,2-dihydro-indazol-3-one;
7-(4′,5′-Difluoro-2′-methyl-biphenyl-4-yloxymethyl)-2-((R)-2,3-dihydroxy-propyl)-1,2-dihydro-indazol-3-one;
7-(4′,5′-Difluoro-2′-methyl-biphenyl-4-yloxymethyl)-2-((S)-2,3-dihydroxy-propyl)-1,2-dihydro-indazol-3-one;
7-(2′-Chloro-4′,5′-difluoro-biphenyl-4-yloxymethyl)-2-((R)-2,3-dihydroxy-propyl)-1,2-dihydro-indazol-3-one; and
7-(2′-Chloro-4′,5′-difluoro-biphenyl-4-yloxymethyl)-2-((S)-2,3-dihydroxy-propyl)-1,2-dihydro-indazol-3-one.

In another embodiment, provided is a pharmaceutical composition, comprising a therapeutically effective amount of a compound according to claim 1, or a pharmaceutically acceptable salt thereof, and a pharmaceutically acceptable carrier and/or adjuvant.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “cycloalkyl” refers to a monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, bornyl, adamantyl, indenyl and the like. In a preferred embodiment, the “cycloalkyl” moieties can optionally be substituted with one, two, three or four substituents with the understanding that said substituents are not, in turn, substituted further unless indicated otherwise in the Examples or claims below. Each substituent can independently be, for example, alkyl, alkoxy, halogen, amino, hydroxyl or oxygen (O═) unless otherwise specifically indicated. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl.

The term “heterocycloalkyl” denotes a mono- or polycyclic alkyl ring, wherein one, two or three of the carbon ring atoms is replaced by a heteroatom such as N, O or S. Examples of heterocycloalkyl groups include, but are not limited to, morpholinyl, thiomorpholinyl, piperazinyl, piperidinyl, pyrrolidinyl, tetrahydropyranyl, tetrahydrofuranyl, 1,3-dioxanyl and the like. The heterocycloalkyl groups may be unsubstituted or substituted and attachment may be through their carbon frame or through their heteroatom(s) where appropriate, with the understanding that said substituents are not, in turn, substituted further unless indicated otherwise in the Examples or claims below.

The term “lower alkyl”, alone or in combination with other groups, refers to a branched or straight-chain alkyl radical of one to nine carbon atoms, preferably one to six carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

The term “aryl” refers to an aromatic mono- or polycarbocyclic radical of 6 to 12 carbon atoms having at least one aromatic ring. Examples of such groups include, but are not limited to, phenyl, napthyl. 1,2,3,4-tetrahydronaphthalene, 1,2-dihydronaphthalene, indanyl, 1H-indenyl and the like.

The alkyl, lower alkyl and aryl groups may be substituted or unsubstituted. When substituted, there will generally be, for example, 1 to 4 substituents present, with the understanding that said substituents are not, in turn, substituted further unless indicated otherwise in the Examples or claims below. Substituents may include, for example, halogen, hydroxy, alkoxy and carboxylic acid.

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. The heteroaryl group may be substituted independently with one, two, or three substituents, with the understanding that said substituents are not, in turn, substituted further unless indicated otherwise in the Examples or claims below. Substituents may include, for example, lower alkyl and halogen.

As used herein, the term “alkoxy” means alkyl-O—; and “alkoyl” means alkyl-CO—. Alkoxy substituent groups or alkoxy-containing substituent groups may be substituted by, for example, one or more alkyl groups with the understanding that said substituents are not, in turn, substituted further unless indicated otherwise in the Examples or claims below.

As used herein, the term “halogen” means a fluorine, chlorine, bromine or iodine radical, preferably a fluorine, chlorine or bromine radical, and more preferably a fluorine or chlorine radical.

Compounds of formula (I) can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminium salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, talc, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day.

It will be appreciated, that the compounds of general formula (I) in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Compounds of the present invention can be prepared beginning with commercially available starting materials and utilizing general synthetic techniques and procedures known to those skilled in the art. Outlined below are reaction schemes suitable for preparing such compounds. Further exemplification can be found in the specific Examples detailed below.

Chemicals may be purchased from companies such as for example Aldrich, Argonaut Technologies, VWR and Lancaster. Chromatography supplies and equipment may be purchased from such companies as for example Analogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography.

Definitions as used herein include:
GS is glycogen synthase,
THF is tetrahydrofuran,
DMF is N,N-dimethylformamide,
DMA is N,N-dimethylacetamide,
DMSO is dimethylsulfoxide,
DCM is dichloromethane,
DME is dimethoxyethane,
MeOH is methanol,
EtOH is ethanol,
NaOH is sodium hydroxide,
TFA is 1,1,1-trifluoroacetic acid,
HOBT is 1-hydroxybenzotriazole,
PyBroP is bromotripyrrolidinophosphonium hexafluorophosphate,
EDCI is 1-[3-(dimethylamino)propyl]-3-ethylcarbodiimide hydrochloride,
DIPEA is diisopropylethylamine,
Boc is tert-butyloxycarbonyl,
NBS is N-bromosuccinimde,
Brine is saturated aqueous sodium chloride solution,
TLC is thin layer chromatography,
RP HPLC is reversed phase high performance liquid chromatography,
HR-MS is high resolution mass spectrometry,
LC-MS is liquid chromatographic mass spectrometry,
RT is room or ambient temperature.

The preparation of N1-alkyl-N1-Boc-hydrazine can be carried out by the direct alkylation of hydrazine with alkyl halide (i) to form N-alkylhydrazine as shown in Scheme 1, below, where R1 group can be alkyl, substituted alkyl, and X can be chloride, bromide or iodide. The treatment of N-alkylhydrazine (ii) with di-tert-butyldicarbonate can provide N1-alkyl-N-1-Boc-hydrazine (iii) as a major desired product with a minor N1-alkyl-N2-Boc-hydrazine. For the preparation of N1-aryl-N-1-Boc-hydrazine, the N1 arylation of N-Boc-hydrazine with aryliodide (iv) under transition metal catalysis condition can provide N1-aryl-N1-Boc-hydrazine by using a similar procedure described in literature (Journal of Organic Chemistry 2009, 74, 4542). As shown in Scheme 1, the aryl group can be aromatic and heteroaromatic groups.

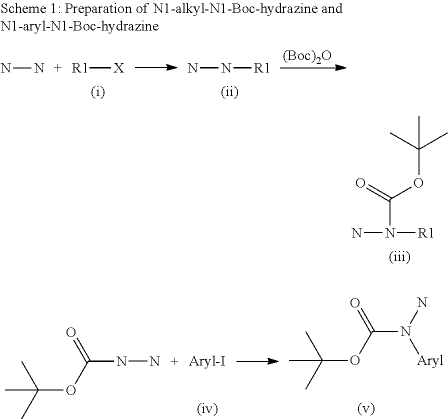

The preparation of 3-bi-phenyloxymethyl-2-bromo-benzoic acid methyl ester is described in Scheme 2, below. The commercially available phenylboronic acid (vi) can be coupled with 4-iodophenol under palladium catalysis conditions to form the bi-aryl-phenol (vii), where R2, R3 and R4 can be fluoro, chloro, methyl or methoxy groups. Alternatively, the required biphenylphenol (vii) can also be prepared through the coupling of 4-hydroxy-arylboronic acid with the corresponding arylbromide under palladium catalysis conditions (Scheme 2). For non-commercially available arylbromides, they can be prepared through aromatic bromination. The bi-aryl-phenol (vii) can be alkylated with 3-bromomethyl-2-bromo-benzoic acid methyl ester (viii) under basic conditions to form 3-bi-phenyloxymethyl-2-bromo-benzoic acid methyl ester (ix).

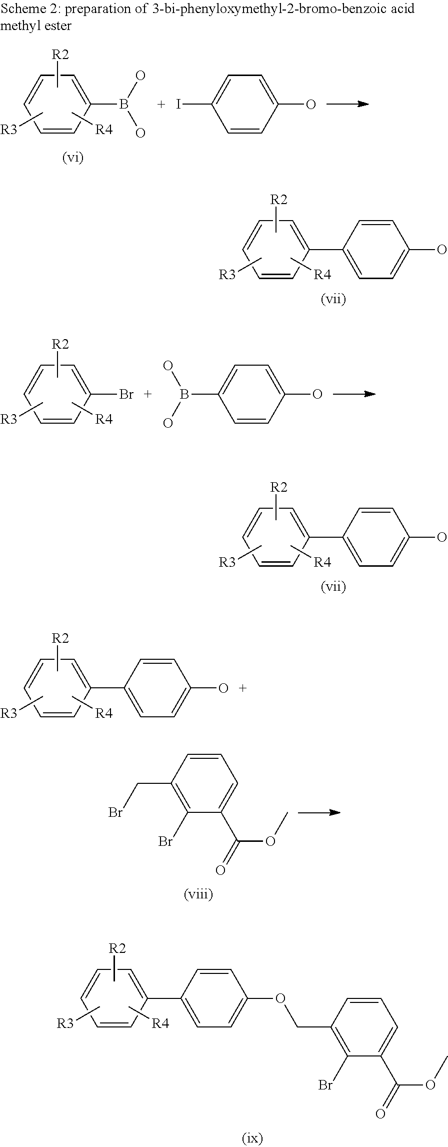

To prepare 7-biaryloxymethyl-2-N-alkyl-indazolone (xi), the corresponding arylbromide (ix) can react with N1-alkyl-N1-Boc-hydrazine in the presence of palladium catalyst, such as palladium acetate, and tri-tert-butyl phosphine ligands to provide the amination product (x). Other palladium catalyst and phosphine ligands can also be applied for the reaction. Treatment of the compound (x) with acid, such as aqueous hydrochloric acid, under refluxing condition can provide the desired 2-N-alkyl-indazolone as described in Scheme 3, below.

To prepare 7-biaryloxymethyl-2-N-aryl-indazolone (xiii), the corresponding arylbromide (ix) can react with N1-aryl-N1-Boc-hydrazine in the presence of palladium catalyst, such as palladium acetate, and tri-tert-butyl phosphine ligands to provide the amination product (xii). Other palladium catalyst and phosphine ligands can also be applied for the reaction. Treatment of the compound (xii) with acid, such as aqueous hydrochloric acid, under refluxing condition can provide the desired 2-N-aryl-indazolone as described in Scheme 3.

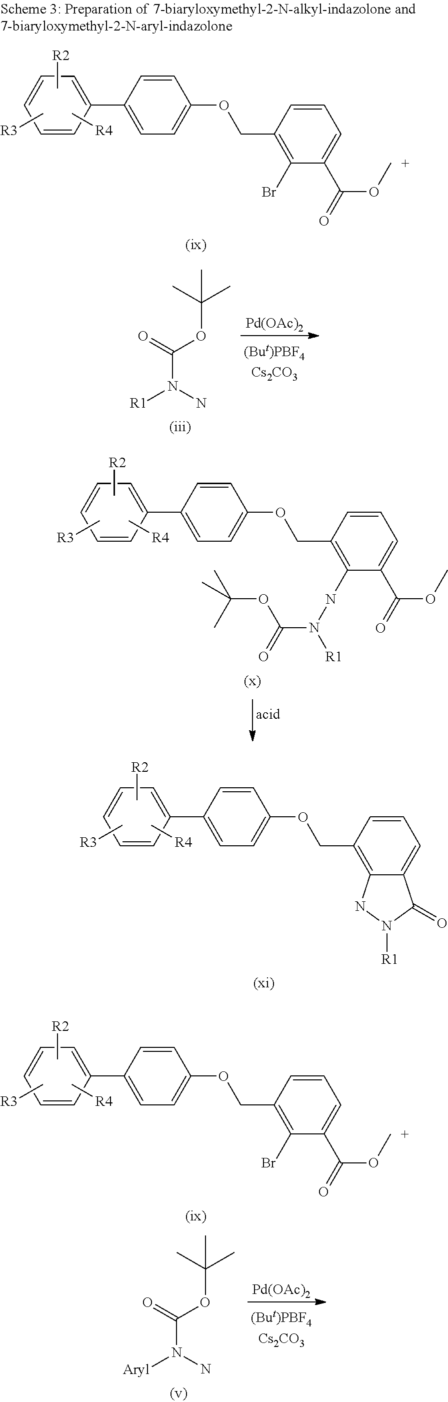

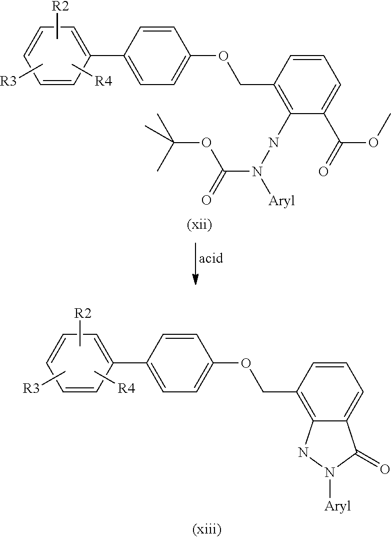

For the preparation of compound (xi) where R1 group contains di-hydroxy, mono-hydroxy or carboxylic acid groups, the synthetic route is described in Scheme 4, below. Compound (xiv), compound (xvi) and compound (xviii) can be made by the amination reaction of the corresponding arylbromide with N1-Boc-N1-alkyl-hydrazine as described in Scheme 3. Heating of compound (xiv) with acid, such as aqueous hydrochloric acid, in organic solvent can de-protect acetonide and undergo cyclization in one step to form the desired diol substituted indazolone derivatives (xv). Both (R))- and (S)-enantiomers of compound (xv) can be prepared under the conditions described in Scheme 4. For chiral compounds, the chiral purity can be obtained through the analysis of chiral chromatography. Likewise, carboxylic acid (xix) can be prepared with the same method as described in Scheme 4. To prepare the mono-hydroxy compound (xvii), an additional step of reduction can be applied as shown Scheme 4. Reducing reagens such as sodium boronhydride can reduce the corresponding aldehyde to the corresponding alcohol (xvii).

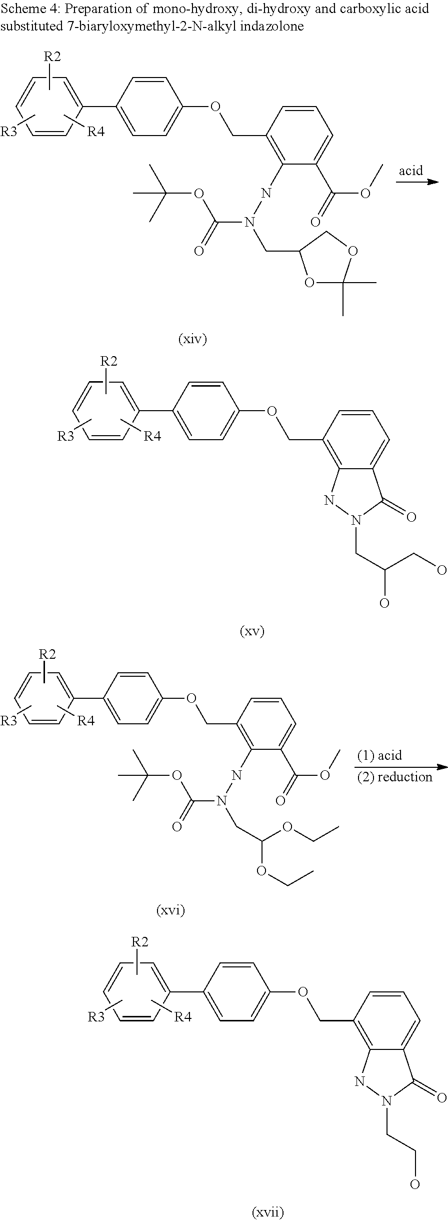

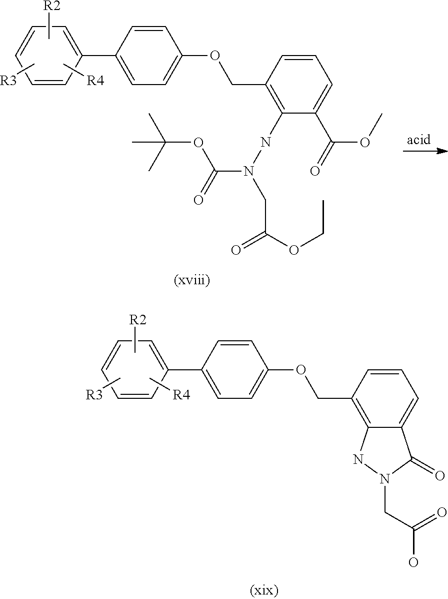

The invention will now be further described in the Examples below, which are intended as an illustration only and do not limit the scope of the invention.

### EXAMPLES

### Part I: Preparation of Preferred Intermediates

### ((R)-2,2-Dimethyl-[1,3]dioxolan-4-ylmethyl)-hydrazine

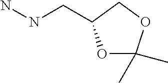

(S)-4-chloromethyl-2,2-dimethyl-1,3-dioxalane (4.6 g, 30.56 mmol) was mixed with anhydrous hydrazine (12 mL). The mixture was stirred at 80° C. for 4.5 hrs to give a homogeneous solution in a sealed tube. The mixture was cooled to room temperature and treated with anhydrous ether (150 mL). The organic layer was quickly separated and dried with sodium sulfate. Solvent was evaporated under reduced pressure (50° C., 40 ton) until most volatile material was removed to give a colorless oil as ((R)-2,2-dimethyl-[1,3]dioxolan-4-ylmethyl)-hydrazine (4.15 g, 93%). ¹H-NMR (CDCl₃) δ ppm 4.30 (qd, J=6.7, 4.2 Hz, 1H), 4.07 (dd, J=8.2, 6.3 Hz, 1H), 3.67 (dd, J=8.2, 6.9 Hz, 1H), 3.23 (br s, 3H), 2.94 (dd, J=12.4, 4.2 Hz, 1H), 2.86 (dd, J=12.4, 7.2 Hz, 1H), 1.42 (s, 3H), 1.36 (s, 3H).

### ((S)-2,2-Dimethyl-[1,3]dioxolan-4-ylmethyl)-hydrazine

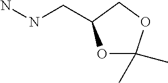

This compound was prepared with the same procedure as described in the preparation of ((R)-2,2-dimethyl-[1,3]dioxolan-4-ylmethyl)-hydrazine by using (R)-4-chloromethyl-2,2-dimethyl-1,3-dioxalane (4.5 g, 29.9 mmol) and anhydrous hydrazine (10 g, 312 mmol). ((S)-2,2-Dimethyl-[1,3]dioxolan-4-ylmethyl)-hydrazine was obtained as a colorless oil (4.13 g, 94.6%). ¹H-NMR (CDCl₃) δ ppm 4.30 (qd, J=6.7, 4.2 Hz, 1H), 4.07 (dd, J=8.2, 6.3 Hz, 1H), 3.67 (dd, J=8.2, 6.9 Hz, 1H), 3.23 (br s, 3H), 2.94 (dd, J=12.4, 4.2 Hz, 1H), 2.86 (dd, J=12.4, 7.2 Hz, 1H), 1.42 (s, 3H), 1.36 (s, 3H).

### N—((R)-2,2-Dimethyl-[1,3]dioxolan-4-ylmethyl)-hydrazinecarboxylic acid tert-butyl ester

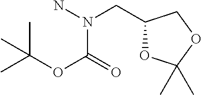

((R)-2,2-dimethyl-[1,3]dioxolan-4-ylmethyl)-hydrazine (4.15 g, 28.4 mmol) was dissolved in methanol (40 mL). The solution was cooled in an ice bath and di-tert-butyl dicarbonate (6.28 g, 28.7 mmol) in methanol (15 mL) was added slowly over 25 minutes. The mixture was stirred at 0° C. for 1 hr and concentrated. The residue was treated with toluene (10 mL) and concentrated to dryness. N—((R)-2,2-Dimethyl-[1,3]dioxolan-4-ylmethyl)-hydrazinecarboxylic acid tert-butyl ester was obtained as colorless oil (6.74 g, 96%). ¹H-NMR (CDCl₃) δ ppm 4.32 (quin, J=6.0 Hz, 1H), 4.05 (dd, J=8.4, 6.0 Hz, 1H), 3.76 (dd, J=8.4, 6.2 Hz, 1H), 3.63 (dd, J=13.7, 6.0 Hz, 1H), 3.43 (dd, J=13.7, 4.4 Hz, 1H), 1.47 (s, 9H), 1.43 (s, 3H), 1.35 (s, 3H).

### N—((S)-2,2-Dimethyl-[1,3]dioxolan-4-ylmethyl)-hydrazinecarboxylic acid tert-butyl ester

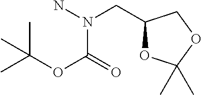

N—((S)-2,2-Dimethyl-[1,3]dioxolan-4-ylmethyl)-hydrazinecarboxylic acid tert-butyl ester was prepared with the same procedure as described for the preparation of N—((R)-2,2-dimethyl-[1,3]dioxolan-4-ylmethyl)-hydrazinecarboxylic acid tert-butyl ester (yield 92.5%). ¹H-NMR (CDCl₃) δ ppm 4.32 (quin, J=6.0 Hz, 1H), 4.05 (dd, J=8.4, 6.0 Hz, 1H), 3.76 (dd, J=8.4, 6.2 Hz, 1H), 3.63 (dd, J=13.7, 6.0 Hz, 1H), 3.43 (dd, J=13.7, 4.4 Hz, 1H), 1.47 (s, 9H), 1.43 (s, 3H), 1.35 (s, 3H).

### 2-Bromo-3-bromomethyl-benzoic acid methyl ester

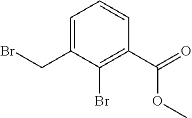

A mixture of 2-bromo-3-methyl-benzoic acid methyl ester (10.20 g, 44.70 mmol), NBS (10.4 g, 58.1 mmol), and V65 catalyst (0.60 g, 2.6 mmol, 6% molar ratio) in dichloromethane (80 mL) was heated with stirring at 42° C. overnight (15 hr). The reaction mixture was filtered through Celite and solvent was removed. The residue was purified through flash column chromatography (200 g silica gel, ethyl acetate in hexanes 0% to 20% over 30 minutes) to afford 2-bromo-3-bromomethyl-benzoic acid methyl ester (8.40 g, 61%) as a clear oil. ¹H NMR (CDCl₃) δ (ppm) 7.61 (dd, J=7.8, 1.6 Hz, 1H), 7.58 (dd, J=7.8, 1.6 Hz, 1H), 7.36 (t, J=7.8 Hz, 1H), 4.68 (s, 2H), 3.95 (s, 3H).

### 2-Bromo-3-(2′,4′,5′-trifluoro-biphenyl-4-yloxymethyl)-benzoic acid methyl ester

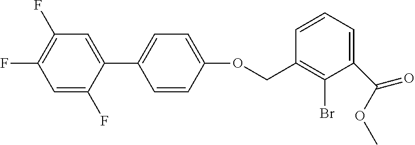

A mixture of 2′,4′,5′-trifluoro-biphenyl-4-ol (2.20 g, 9.8 mmol), 2-bromo-3-bromomethyl-benzoic acid methyl ester (3.01 g, 9.8 mmol), and potassium carbonate (2.7 g, 19.6 mmol) in acetone (100 mL) was heated to 50° C. and stirred for 6 h. The reaction mixture was diluted with 100 mL of ethyl acetate and filtered through Celite. Solvents were removed and the residue was purified by crystallization in methanol to afford 2-bromo-3-(2′,4′,5′-trifluoro-biphenyl-4-yloxymethyl)-benzoic acid methyl ester as a white solid (4.3 g, 97%). ¹H NMR (DMSO-d₆) δ (ppm) 7.75 (dd, J=7.5, 1.5 Hz, 1H), 7.48-7.73 (m, 6H), 7.14 (d, J=8.8 Hz, 2H), 5.25 (s, 2H), 3.88 (s, 3H).

### 2-Bromo-3-(4′,5′-difluoro-2′-methoxy-biphenyl-4-yloxymethyl)-benzoic acid methyl ester

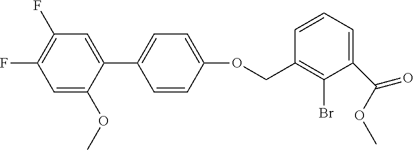

With a method similar to that used for the preparation of 2-bromo-3-(2′,4′,5′-trifluoro-biphenyl-4-yloxymethyl)-benzoic acid methyl ester, this compound was prepared from 4′,5′-difluoro-2′-methoxy-biphenyl-4-ol and 2-bromo-3-bromomethyl-benzoic acid methyl ester. ¹H NMR (DMSO-d₆) δ (ppm) 7.75 (dd, J=7.5, 1.8 Hz, 1H), 7.66 (dd, J=7.5, 1.8 Hz, 1H), 7.54 (t, J=7.5 Hz, 1H), 7.43 (d, J=8.8 Hz, 2H), 7.37 (dd, J=11.5, 9.4 Hz, 1H), 7.24 (dd, J=13.0, 7.2 Hz, 1H), 7.06 (d, J=8.8 Hz, 2H), 5.22 (s, 2H), 3.88 (s, 3H), 3.76 (s, 3H).

### 2-Bromo-3-(4′,5′-difluoro-2′-methyl-biphenyl-4-yloxymethyl)-benzoic acid methyl ester

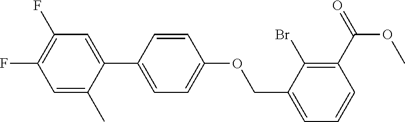

With a method similar to that used for the preparation of 2-bromo-3-(2′,4′,5′-trifluoro-biphenyl-4-yloxymethyl)-benzoic acid methyl ester, this compound was prepared from 4′,5′-difluoro-2′-methyl-biphenyl-4-ol and 2-bromo-3-bromomethyl-benzoic acid methyl ester. The 4′,5′-difluoro-2′-methyl-biphenyl-4-ol was prepared from 4,5-difluoro-2-methyl-1-bromobenzene and 4-hydroxyphenylboronic acid. ¹H NMR, ¹H NMR (CDCl₃) δ (ppm) 7.73 (dd, J=7.6, 1.6 Hz, 1H), 7.67 (dd, J=7.6, 1.6 Hz, 1H), 7.37-7.48 (m, 1H), 7.22 (d, J=8.8 Hz, 2H), 7.03 (d, J=8.8 Hz, 2H), 6.97-7.10 (m, 2H), 5.24 (s, 2H), 3.97 (s, 3H), 2.21 (s, 3H).

### 2-Bromo-3-(2′-chloro-4′,5′-difluoro-biphenyl-4-yloxymethyl)-benzoic acid methyl ester

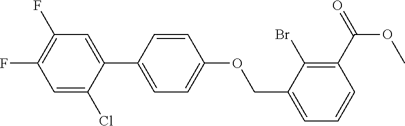

With a method similar to that used for the preparation of 2-bromo-3-(2′,4′,5′-trifluoro-biphenyl-4-yloxymethyl)-benzoic acid methyl ester, this compound was prepared from 2′-chloro-4′,5′-difluoro-biphenyl-4-ol and 2-bromo-3-bromomethyl-benzoic acid methyl ester. ¹H-NMR (CDCl₃) δ ppm 7.70-7.75 (m, 1H), 7.67 (dd, J=7.6, 1.7 Hz, 1H), 7.43 (t, J=7.6 Hz, 1H), 7.36 (d, J=9.1 Hz, 2H), 7.28-7.35 (m, 1H), 7.17 (dd, J=10.7, 8.3 Hz, 1H), 7.05 (d, J=9.1 Hz, 2H), 5.25 (s, 2H), 3.97 (s, 3H).

### N-Pyridin-3-yl-hydrazinecarboxylic acid tert-butyl ester

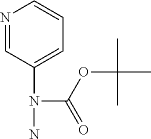

This compound was prepared according procedures described in literature (Journal of Organic Chemistry 2009, 74, 4542-4546). Treatment of tert-butylhydrazine carboxylate with 3-iodopyridine and cesium carbonate in the presence of catalytic amount of copper iodide in DMSO provided N-pyridin-3-yl-hydrazinecarboxylic acid tert-butyl ester. ¹H NMR (DMSO-d₆) δ (ppm) 8.77 (br. s., 1H), 8.29 (br. s., 1H), 7.85 (d, J=8.2 Hz, 1H), 7.36 (br. s., 1H), 5.15 (br. s., 2H), 1.46 (s, 9H).

### N-(4-Fluoro-phenyl)-hydrazinecarboxylic acid tert butyl ester

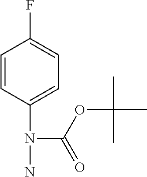

With the same method described for the preparation of N-pyridin-3-yl-hydrazinecarboxylic acid tert-butyl ester, N-(4-fluoro-phenyl)-hydrazinecarboxylic acid tert butyl ester was prepared from tert-butylhydrazine carboxylate and 4-fluoro-1-idodobenzene. ¹H NMR (DMSO-d₆) δ (ppm) 7.44 (dd, J=9.0, 5.1 Hz, 2H), 7.10 (t, J=9.0 Hz, 2H), 5.07 (s, 2H), 1.43 (s, 9H).

### (N-tert-Butoxycarbonyl-hydrazino)-acetic acid ethyl ester

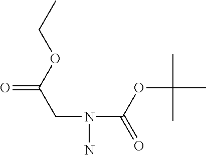

A mixture of hydrazino-acetic acid ethyl ester HCl salt (4.04 g, 26.13 mmol), di-tert-butyl dicarbonate (5.70 g, 26.13 mmol), and N-methylmorpholine (2.87 g, 28.42 mmol) was stirred in ethanol (25 mL) and water (25 mL) under ice bath. The reaction was warmed to room temperature and stirred for 3 h. The mixture was quenched with saturated ammonium chloride (100 mL) and the aqueous solution was extracted twice with 100 mL of diethyl ether. The ether layer was washed with water and dried with magnesium sulfate. Solvents were removed to afford (N-tert-butoxycarbonyl-hydrazino)-acetic acid ethyl ester (5.40 g, 95% yield) as colorless oil. ¹H NMR (DMSO-d₆) δ (ppm) 4.55 (s, 2H), 4.03-4.20 (m, 2H), 4.01 (s, 2H), 1.38 (s, 9H), 1.20 (t, J=7.1 Hz, 3H).

### Part II: Preparation of Preferred Embodiments of the Invention

### Example 1

### 7-(4′,5′-Difluoro-2′-methyl-biphenyl-4-yloxymethyl)-2-methyl-1,2-dihydro-indazol-3-one

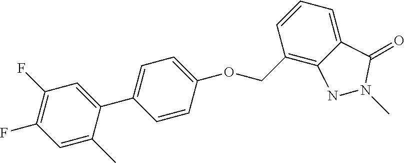

A mixture of 2-bromo-3-(4′,5′-difluoro-2′-methyl-biphenyl-4-yloxymethyl)-benzoic acid methyl ester (420 mg, 0.94 mmol), N-methyl-hydrazinecarboxylic acid tert-butyl ester (223 mg, 1.53 mmol), cesium carbonate (710 mg, 2.18 mmol), palladium acetate (29.0 mg, 0.13 mmol) and tri-t-butylphosphonium tetrafluoroborate (53.0 mg, 0.18 mmol) in toluene (9 mL) was heated to 110° C. in a sealed tube flushed with argon and stirred for 5 h. The reaction mixture was diluted with ethyl acetate (25 mL) and filtered through Celite. Solvents were removed and the residue was purified through flash column chromatography (silica gel 40 g, 0% to 25% of ethyl acetate in hexanes) to afford 2-(N′-tert-butoxycarbonyl-N′-methyl-hydrazino)-3-(4′,5′-difluoro-2′-methyl-biphenyl-4-yloxymethyl)-benzoic acid methyl ester as an amorphous solid (440 mg, 92% yield).

¹H-NMR (DMSO-d₆) δ (ppm) 8.79 (s, 1H), 7.64 (d, J=7.5 Hz, 1H), 7.60 (d, J=7.5 Hz, 1H), 7.37 (dd, J=11.8, 8.5 Hz, 1H), 7.29 (d, J=8.8 Hz, 2H), 7.23 (dd, J=11.5, 8.5 Hz, 1H), 7.04 (d, J=8.8 Hz, 2H), 6.95 (t, J=7.5 Hz, 1H), 5.05 (s, 2H), 3.82 (s, 3H), 3.03 (s, 3H), 2.19 (s, 3H), 1.20 (s, 9H).

To a solution of 2-(N′-tert-butoxycarbonyl-N′-methyl-hydrazino)-3-(4′,5′-difluoro-2′-methyl-biphenyl-4-yloxymethyl)-benzoic acid methyl ester (440 mg, 0.86 mmol) in THF (10 mL) was added hydrochloric (3M, 15 mL) and the mixture was stirred for 2 h at reflux. The reaction mixture was concentrated and the residue was extracted with ethyl acetate (50 mL). The organic layer was washed with water (50 mL) and brine (50 mL), dried with anhydrous sodium sulfate. After evaporation of solvents, the crude product was triturated with ether and filtered to afford 7-(4′,5′-difluoro-2′-methyl-biphenyl-4-yloxymethyl)-2-methyl-indazol-3-one as a white solid (203.0 mg, 62% yield). HRMS (ES+) calcd for C₂₂H₁₈F₂N₂O₂ (M+H) 381.1409, obsd 381.1409; ¹H NMR (DMSO-d₆) δ (ppm) 10.41 (s, 1H), 7.64 (d, J=8.2 Hz, 1H), 7.61 (d, J=8.2 Hz, 1H), 7.38 (dd, J=11.9, 8.6 Hz, 1H), 7.31 (d, J=8.8 Hz, 2H), 7.23 (dd, J=11.8, 8.5 Hz, 1H), 7.12 (d, J=8.8 Hz, 2H), 7.09-7.17 (m, 1H), 5.24 (s, 2H), 3.41 (s, 3H), 2.20 (s, 3H).

### Example 2

### 2-Methyl-7-(2′,4′,5′-trifluoro-biphenyl-4-yloxymethyl)-1,2-dihydro-indazol-3-one

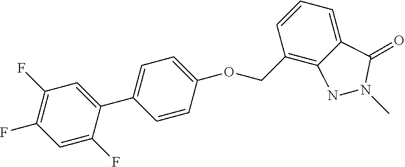

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-(N′-tert-butoxycarbonyl-N′-methyl-hydrazino)-3-(2′,4′,5′-trifluoro-biphenyl-4-yloxymethyl)-benzoic acid methyl ester was prepared from 2-bromo-3-(2′,4′,5′-trifluoro-biphenyl-4-yloxymethyl)-benzoic acid methyl ester and N-methyl-hydrazinecarboxylic acid tert-butyl ester. ¹H NMR (DMSO-d₆) δ (ppm) 8.80 (br. s., 1H), 7.55-7.73 (m, 4H), 7.51 (d, J=8.8 Hz, 2H), 7.08 (d, J=8.8 Hz, 2H), 6.95 (t, J=7.7 Hz, 1H), 5.07 (s, 2H), 3.82 (s, 3H), 3.02 (s, 3H), 1.21 (s, 9H).

2-Methyl-7-(2′,4′,5′-trifluoro-biphenyl-4-yloxymethyl)-1,2-dihydro-indazol-3-one was obtained by refluxing 2-(N′-tert-butoxycarbonyl-N′-methyl-hydrazino)-3-(2′,4′,5′-trifluoro-biphenyl-4-yloxymethyl)-benzoic acid methyl ester with hydrochloric acid in THF as described in the preparation of 7-(4′,5′-difluoro-2′-methyl-biphenyl-4-yloxymethyl)-2-methyl-indazol-3-one. HRMS-ES(+) calcd for C₂₁H₁₅F₃N₂O₂ (M+H) 385.1159, obsd 385.1159; ¹H NMR (DMSO-d₆) δ (ppm) 10.43 (s, 1H), 7.56-7.74 (m, 4H), 7.52 (dd, J=8.8, 1.2 Hz, 2H), 7.17 (d, J=8.8 Hz, 2H), 7.08-7.15 (m, 1H), 5.26 (s, 2H), 3.41 (s, 3H).

### Example 3

### 7-(4′,5′-Difluoro-2′-methoxy-biphenyl-4-yloxymethyl)-2-methyl-1,2-dihydro-indazol-3-one

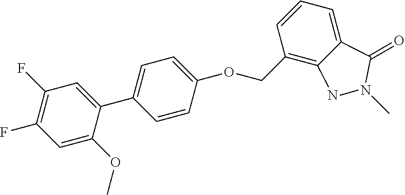

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-(N′-tert-butoxycarbonyl-N′-methyl-hydrazino)-3-(4′,5-difluoro-2′-methoxy-biphenyl-4-yloxymethyl)-benzoic acid methyl ester was prepared from 2-bromo-3-(4′,5′-difluoro-2′-methoxy-biphenyl-4-yloxymethyl)-benzoic acid methyl ester and N-methyl-hydrazinecarboxylic acid tert-butyl ester. ¹H NMR (DMSO-d₆) δ (ppm) 8.80 (s, 1H), 7.64 (d, J=7.7 Hz, 1H), 7.59 (d, J=7.7 Hz, 1H), 7.42 (d, J=8.8 Hz, 2H), 7.35 (dd, J=11.5, 9.4 Hz, 1H), 7.23 (dd, J=13.1, 7.1 Hz, 1H), 7.00 (d, J=8.8 Hz, 2H), 6.95 (t, J=7.7 Hz, 1H), 5.05 (s, 2H), 3.82 (s, 3H), 3.75 (s, 3H), 3.03 (s, 3H), 1.21 (s, 9H)

7-(4′,5′-Difluoro-2′-methoxy-biphenyl-4-yloxymethyl)-2-methyl-1,2-dihydro-indazol-3-one was obtained by refluxing 2-(N′-tert-butoxycarbonyl-N′-methyl-hydrazino)-3-(4′,5′-difluoro-2′-methoxy-biphenyl-4-yloxymethyl)-benzoic acid methyl ester with hydrochloric acid in THF as described in the preparation of 7-(4′,5′-difluoro-2′-methyl-biphenyl-4-yloxymethyl)-2-methyl-indazol-3-one. HRMS-ES(+) calcd for C₂₂H₁₈F₂N₂O₃ (M+H) 397.1358, obsd 397.1356; ¹H NMR (DMSO-d₆) δ (ppm) 10.41 (s, 1H), 7.63 (d, J=8.2 Hz, 1H), 7.60 (d, J=7.5 Hz, 1H), 7.43 (d, J=8.6 Hz, 2H), 7.36 (dd, J=11.2, 9.4 Hz, 1H), 7.24 (dd, J=13.0, 6.9 Hz, 1H), 7.10-7.16 (m, 1H), 7.09 (d, J=8.6 Hz, 2H), 5.24 (s, 2H), 3.76 (s, 3H), 3.41 (s, 3H).

### Example 4

### [3-oxo-7-(2′,4′,5′-trifluoro-biphenyl-4-yloxymethyl)-1,3-dihydro-indazol-2-yl]-acetic acid

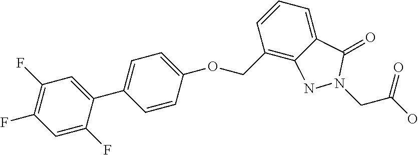

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-(N′-tert-butoxycarbonyl-N′-ethoxycarbonylmethyl-hydrazino)-3-(2′,4′,5′-trifluoro-biphenyl-4-yloxymethyl)-benzoic acid methyl ester was prepared from 2-bromo-3-(2′,4′,5′-trifluoro-biphenyl-4-yloxymethyl)-benzoic acid methyl ester and (N-tert-butoxycarbonyl-hydrazino)-acetic acid ethyl ester. ¹H-NMR (DMSO-d₆) δ (ppm) 9.52 (br. s., 1H), 7.86 (d, J=7.8 Hz, 1H), 7.57-7.74 (m, 3H), 7.51 (d, J=8.8 Hz, 2H), 7.08 (d, J=8.8 Hz, 2H), 7.02 (t, J=7.8 Hz, 1H), 5.10 (s, 2H), 4.27 (br. s., 2H), 4.18 (q, J=7.0 Hz, 2H), 3.86 (s, 3H), 1.23 (t, J=7.0 Hz, 3H), 1.17 (br. s, 9H).

[3-oxo-7-(2′,4′,5′-trifluoro-biphenyl-4-yloxymethyl)-1,3-dihydro-indazol-2-yl]-acetic acid was obtained by refluxing 2-(N′-tert-butoxycarbonyl-N′-ethoxycarbonylmethyl-hydrazino)-3-(2′,4′,5′-trifluoro-biphenyl-4-yloxymethyl)-benzoic acid methyl ester with hydrochloric acid in THF as described in the preparation of 7-(4′,5′-difluoro-2′-methyl-biphenyl-4-yloxymethyl)-2-methyl-indazol-3-one. LRMS-ES(+) calcd for C₂₂H₁₅F₃N₂O₄ (M+H) 429.1, obsd 429.1; ¹H-NMR (DMSO-d₆) δ (ppm) 13.09 (br. s., 1H), 10.33 (br. s., 1H), 7.58-7.72 (m, 4H), 7.52 (dd, J=8.8, 1.5 Hz, 2H), 7.16 (d, J=8.8 Hz, 2H), 7.09-7.22 (m, 1H), 5.27 (s, 2H), 4.56 (s, 2H)

### Example 5

### [7-(4′,5′-Difluoro-2′-methoxy-biphenyl-4-yloxymethyl)-3-oxo-1,3-dihydro-indazol-2-yl]-acetic acid

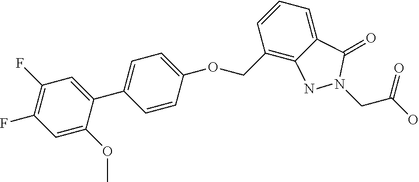

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-(N′-tert-butoxycarbonyl-N′-ethoxycarbonylmethyl-hydrazino)-3-(4′,5′-difluoro-2′-methoxy-biphenyl-4-yloxymethyl)-benzoic acid methyl ester was prepared from 2-bromo-3-(2′-methoxy-4′,5′-difluoro-biphenyl-4-yloxymethyl)-benzoic acid methyl ester and (N-tert-butoxycarbonyl-hydrazino)-acetic acid ethyl ester. ¹H-NMR (DMSO-d₆) δ (ppm) 9.50 (br. s., 1H), 7.85 (d, J=7.8 Hz, 1H), 7.66 (d, J=7.2 Hz, 1H), 7.42 (d, J=8.8 Hz, 2H), 7.36 (dd, J=11.6, 9.5 Hz, 1H), 7.23 (dd, J=13.1, 7.1 Hz, 1H), 6.96-7.07 (m, 1H), 7.00 (d, J=8.8 Hz, 2H), 5.08 (s, 2H), 4.28 (br. s., 2H), 4.18 (q, J=7.0 Hz, 2H), 3.86 (s, 3H), 3.75 (s, 3H), 1.23 (t, J=7.0 Hz, 3H), 1.17 (br. s, 9H).

[7-(4′,5′-Difluoro-2′-methoxy-biphenyl-4-yloxymethyl)-3-oxo-1,3-dihydro-indazol-2-yl]-acetic acid was prepared by refluxing 2-(N′-tert-butoxycarbonyl-N′-ethoxycarbonylmethyl-hydrazino)-3-(4′,5′-difluoro-2′-methoxy-biphenyl-4-yloxymethyl)-benzoic acid methyl ester with hydrochloric acid in THF as described in the previous examples. HRMS (ES+) calcd for C₂₃H₁₈F₂N₂O₅ (M+H) 441.1257, obsd 441.1256; ¹H NMR (DMSO-d₆) δ 13.09 (br. s., 1H), 10.33 (br. s., 1H), 7.67 (d, J=7.5 Hz, 1H), 7.64 (d, J=7.5 Hz, 1H), 7.42 (d, J=8.8 Hz, 2H), 7.35 (dd, J=11.5, 9.4 Hz, 1H), 7.24 (dd, J=13.0, 6.9 Hz, 1H), 7.15 (t, J=7.5 Hz, 1H), 7.09 (d, J=8.8 Hz, 2H), 5.24 (s, 2H), 4.55 (s, 2H), 3.75 (s, 3H).

### Example 6

### 2-(2-Methoxy-ethyl)-7-(2′,4′,5′-trifluoro-biphenyl-4-yloxymethyl)-1,2-dihydro-indazol-3-one Hydrochloride

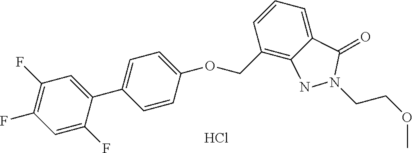

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-[N′-tert-butoxycarbonyl-N′-(2-methoxy-ethyl)-hydrazino]-3-(2′,4′,5′-trifluoro-biphenyl-4-yloxymethyl)-benzoic acid methyl ester was prepared from 2-bromo-3-(2′,4′,5′-trifluoro-biphenyl-4-yloxymethyl)-benzoic acid methyl ester and N-(2-methoxy-ethyl)-hydrazine carboxylic acid tert-butyl ester. LRMS calcd for C₂₉H₃₁F₃N₂O₆ (M+H) 561.0, obsd 561.0

2-(2-Methoxy-ethyl)-7-(2′,4′,5′-trifluoro-biphenyl-4-yloxymethyl)-1,2-dihydro-indazol-3-one was prepared by refluxing 2-[N′-tert-butoxycarbonyl-N′-(2-methoxy-ethyl)-hydrazino]-3-(2′,4′,5′-trifluoro-biphenyl-4-yloxymethyl)-benzoic acid methyl ester and hydrochloric acid in THF as described in the previous example. The final compound was obtained after conversion to a hydrochloride salt. HRMS-ES(+) calcd for C₂₃H₁₉F₃N₂O₃ (M+H) 429.1421, obsd 429.1420; ¹H NMR (DMSO-d₆) δ: 10.58 (br. s, 1H), 7.56-7.73 (m, 4H), 7.52 (dd, J=8.7, 1.4 Hz, 2H), 7.16 (d, J=8.7 Hz, 2H), 7.09-7.17 (m, 1H), 5.30 (s, 2H), 4.00 (t, J=5.6 Hz, 2H), 3.66 (t, J=5.6 Hz, 2H), 3.26 (s, 3H)

### Example 7

### 7-(4′,5′-Difluoro-2′-methoxy-biphenyl-4-yloxymethyl)-2-(2-methoxy-ethyl)-1,2-dihydro-indazol-3-one

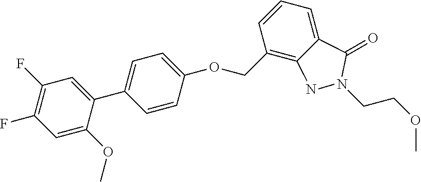

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-[N′-tert-butoxycarbonyl-N′-(2-methoxy-ethyl)-hydrazino]-3-(4′,5′-difluoro-2′-methoxy-biphenyl-4-yloxymethyl)-benzoic acid methyl ester was prepared from 2-bromo-3-(2′-methoxy-4′,5′-difluoro-biphenyl-4-yloxymethyl)-benzoic acid methyl ester and N-(2-methoxy-ethyl)-hydrazine carboxylic acid tert-butyl ester. LC-MS showed a single peak with retention time of 4.38 min. LRMS calcd for C₃₀H₃₄F₂N₂O₇ (M+H) 573.1, obsd 573.1.

7-(4′,5′-Difluoro-2′-methoxy-biphenyl-4-yloxymethyl)-2-(2-methoxy-ethyl)-1,2-dihydro-indazol-3-one was prepared by refluxing 2-[N′-tert-butoxycarbonyl-N′-(2-methoxy-ethyl)-hydrazino]-3-(4′,5′-difluoro-2′-methoxy-biphenyl-4-yloxymethyl)-benzoic acid methyl ester with hydrochloric acid in THF as described in the previous example. HRMS-ES(+) calcd for C₂₄H₂₂F₂N₂O₄ (M+H) 441.1621, obsd 441.1619; ¹H NMR (DMSO-d₆) δ (PPM) 10.21 (s, 1 H), 7.62 (t, J=7.3 Hz, 2 H), 7.43 (d, J=8.8 Hz, 2 H), 7.36 (dd, J=11.5, 9.4 Hz, 1 H), 7.24 (dd, J=13.0, 7.2 Hz, 1 H), 7.13 (t, J=7.3 Hz, 1 H), 7.09 (d, J=8.8 Hz, 2 H), 5.26 (s, 2 H), 3.99 (t, J=5.6 Hz, 2 H), 3.75 (s, 3 H), 3.65 (t, J=5.6 Hz, 2 H), 3.26 (s, 3 H).

### Example 8

### 2-(2-Hydroxy-ethyl)-7-(2′,4′,5′-trifluoro-biphenyl-4-yloxymethyl)-1,2-dihydro-indazol-3-one

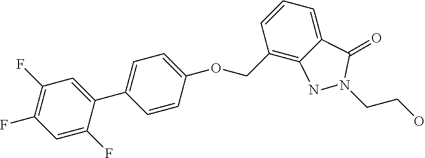

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-[N′-tert-butoxycarbonyl-N′-(2,2-diethoxy-ethyl)-hydrazino]-3-(2′,4′,5′-trifluoro-biphenyl-4-yloxymethyl)-benzoic acid methyl ester was prepared from 2-bromo-3-(2′-methoxy-4′,5′-difluoro-biphenyl-4-yloxymethyl)-benzoic acid methyl ester and N-(2,2-diethoxy-ethyl)-hydrazinecarboxylic acid tert-butyl ester. ¹H-NMR (DMSO-d₆) δ (ppm) 9.26 (br. s., 1H), 7.83 (d, J=7.7 Hz, 1H), 7.58-7.73 (m, 3H), 7.51 (d, J=8.8 Hz, 2H), 7.06 (d, J=8.8 Hz, 2H), 6.98 (t, J=7.7 Hz, 1H), 5.04 (s, 2H), 4.71 (t, J=5.7 Hz, 1H), 3.84 (s, 3H), 3.56-3.75 (m, 2H), 3.39-3.60 (m, 4H), 1.20 (br. s., 9H), 1.13 (t, J=6.9 Hz, 6H).

A mixture of 2-[N′-tert-butoxycarbonyl-N′-(2,2-diethoxy-ethyl)-hydrazino]-3-(2′,4′,5′-trifluoro-biphenyl-4-yloxymethyl)-benzoic acid methyl ester (700 mg, 1.10 mmol), and 3M HCl (15 mL) in THF (10 mL) was stirred and refluxed for 2 h. The reaction mixture was diluted with ethyl acetate (50 mL) and washed with water (100 mL). The organic layer was further washed with brine (50 mL), dried with anhydrous sodium sulfate and solvents were removed to yield [3-oxo-7-(2′,4′,5′-trifluoro-biphenyl-4-yloxymethyl)-1,3-dihydro-indazol-2-yl]-acetaldehyde as a crude intermediate. The crude intermediate was treated with sodium borohydride (40 mg, 1.1 mmol) in methanol (2 mL) and THF (1 mL). The solution was stirred for 1 h at room temperature. The reaction mixture was diluted with ethyl acetate (50 mL) and washed with hydrochloric acid (1N, 50 mL). The organic layer was washed with water (50 mL), brine (50 mL), dried with anhydrous sodium sulfate and the solvent was removed. The residue was purified on a flash chromatography column (silica gel 12 g) with ethyl acetate in hexanes (40% to 100%) to afford 2-(2-hydroxy-ethyl)-7-(2′,4′,5′-trifluoro-biphenyl-4-yloxymethyl)-1,2-dihydro-indazol-3-one (130.0 mg, 29% yield) as a white solid. HRMS-ES(+) calcd for C₂₂H₁₇F₃N₂O₃ (M+H) 415.1264, obsd 415.1264; ¹H NMR (DMSO-d₆) δ 10.24 (br. s., 1H), 7.56-7.76 (m, 4H), 7.52 (d, J=7.2 Hz, 2H), 7.07-7.21 (m, 3H), 5.28 (s, 2H), 4.99 (br. s., 1H), 3.90 (t, J=5.7 Hz, 2H), 3.70 (br. s., 2H).

### Example 9

### [7-(4′,5′-Difluoro-2′-methyl-biphenyl-4-yloxymethyl)-3-oxo-1,3-dihydro-indazol-2-yl]acetic acid Hydrochloride

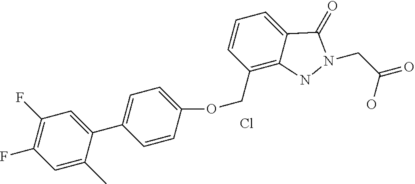

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-(N′-tert-butoxycarbonyl-N′-ethoxycarbonylmethyl-hydrazino)-3-(4′,5′-difluoro-2′-methyl-biphenyl-4-yloxymethyl)-benzoic acid methyl ester was prepared from 2-bromo-3-(4′,5′-difluoro-2′-methyl-biphenyl-4-yloxymethyl)-benzoic acid methyl ester and (N-tert-butoxycarbonyl-hydrazino)-acetic acid ethyl ester. ¹H-NMR (DMSO-d₆) δ (ppm) 9.49 (br. s., 1H), 7.86 (d, J=7.2 Hz, 1H), 7.68 (d, J=7.2 Hz, 1H), 7.38 (dd, J=11.8, 8.5 Hz, 1H), 7.29 (d, J=8.8 Hz, 2H), 7.24 (dd, J=12.1, 8.2 Hz, 1H), 7.04 (d, J=8.8 Hz, 2H), 6.99-7.08 (m, 1H), 5.09 (s, 2H), 4.28 (br. s., 2H), 4.18 (q, J=7.0 Hz, 2H), 3.86 (s, 3H), 2.20 (s, 3H), 1.24 (t, J=7.0 Hz, 3H), 1.18 (br. s, 9H).

[7-(4′,5′-Difluoro-2′-methyl-biphenyl-4-yloxymethyl)-3-oxo-1,3-dihydro-indazol-2-yl]-acetic acid was prepared by refluxing 2-(N′-tert-butoxycarbonyl-N′-ethoxycarbonylmethyl-hydrazino)-3-(4′,5′-difluoro-2′-methyl-biphenyl-4-yloxymethyl)-benzoic acid methyl ester with hydrochloric acid in THF as described in the previous example. HRMS (ES+) calcd for C₂₃H₁₈F₂N₂O₄ (M+H) 425.1308, obsd 425.1307; ¹H-NMR (DMSO-d₆) δ (ppm) 12.61 (br s, 1H), 10.35 (br s, 1H), 7.68 (d, J=7.6 Hz, 1H), 7.65 (d, J=7.6 Hz, 1H), 7.38 (dd, J=12.1, 8.5 Hz, 1H), 7.30 (d, J=8.5 Hz, 2H), 7.23 (dd, J=11.8, 8.5 Hz, 1H), 7.08-7.17 (m, 3H), 5.25 (s, 2H), 4.56 (s, 2H), 2.19 (s, 3H).

### Example 10

### 7-(4′,5′-Difluoro-2′-methyl-biphenyl-4-yloxymethyl)-2-(4-fluoro-phenyl)-1,2-dihydro-indazol-3-one

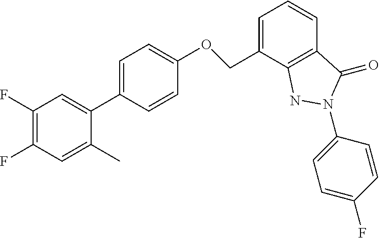

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-[N-tert-butoxycarbonyl-N′-(4-fluoro-phenyl)-hydrazino]-3-(4′,5′-difluoro-2′-methyl-biphenyl-4-yloxymethyl)-benzoic acid methyl ester was prepared from 2-bromo-3-(2′-methyl-4′,5′-difluoro-biphenyl-4-yloxymethyl)-benzoic acid methyl ester and N-(4-fluoro-phenyl)-hydrazinecarboxylic acid tert-butyl ester. ¹H-NMR (DMSO-d₆) δ (ppm) 9.48 (s, 1H), 7.77 (dd, J=7.7, 1.3 Hz, 1H), 7.67 (dd, J=7.7, 1.3 Hz, 1H), 7.57 (dd, J=9.0, 5.0 Hz, 2H), 7.37 (dd, J=12.1, 8.5 Hz, 1H), 7.22 (d, J=8.8 Hz, 2H), 7.18-7.26 (m, 1H), 7.11 (t, J=9.0 Hz, 2H), 7.05 (t, J=7.7 Hz, 1H), 6.82 (d, J=8.8 Hz, 2H), 4.92 (s, 2H), 3.79 (s, 3H), 2.18 (s, 3H), 1.23 (s, 9H)

7-(4′,5′-Difluoro-2′-methyl-biphenyl-4-yloxymethyl)-2-(4-fluoro-phenyl)-1,2-dihydro-indazol-3-one was prepared by refluxing 2-[N-tert-butoxycarbonyl-N′-(4-fluoro-phenyl)-hydrazino]-3-(4′,5′-difluoro-2′-methyl-biphenyl-4-yloxymethyl)-benzoic acid methyl ester with hydrochloric acid in THF as described in the previous example. LRMS calcd for C₂₇H₁₉F₃N₂O₂ (M+H) 461.1, obsd 461.0; ¹H-NMR (DMSO-d₆) δ (ppm) 10.73 (s, 1H), 7.95 (dd, J=9.2, 5.0 Hz, 2H), 7.75 (d, J=7.8 Hz, 1H), 7.73 (d, J=7.8 Hz, 1H), 7.40 (d, J=9.0 Hz, 2H), 7.19-7.38 (m, 5H), 7.15 (d, J=9.0 Hz, 2H), 5.34 (s, 2H), 2.20 (s, 3H)

### Example 11

### 7-(4′,5′-Difluoro-2′-methoxy-biphenyl-4-yloxymethyl)-2-(4-fluoro-phenyl)-1,2-dihydro-indazol-3-one

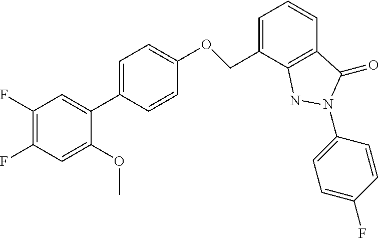

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-[N′-tert-butoxycarbonyl-N′-(4-fluoro-phenyl)-hydrazino]-3-(4′,5′-difluoro-2′-methoxy-biphenyl-4-yloxymethyl)-benzoic acid methyl ester was prepared from 2-bromo-3-(2′-methoxy-4′,5′-difluoro-biphenyl-4-yloxymethyl)-benzoic acid methyl ester and N-(4-fluoro-phenyl)-hydrazinecarboxylic acid tert-butyl ester. ¹H-NMR (DMSO-d₆) δ (ppm) 9.48 (s, 1H), 7.76 (dd, J=7.7, 1.4 Hz, 1H), 7.66 (dd, J=7.7, 1.4 Hz, 1H), 7.58 (dd, J=9.0, 5.0 Hz, 2H), 7.35 (d, J=8.8 Hz, 2H), 7.33-7.39 (m, 1H), 7.24 (dd, J=13.0, 6.9 Hz, 1H), 7.14 (t, J=9.0 Hz, 2H), 7.05 (t, J=7.7 Hz, 1H), 6.79 (d, J=8.8 Hz, 2H), 4.92 (s, 2H), 3.80 (s, 3H), 3.76 (s, 3H), 1.25 (s, 9H)

7-(4′,5′-Difluoro-2′-methoxy-biphenyl-4-yloxymethyl)-2-(4-fluoro-phenyl)-1,2-dihydro-indazol-3-one was prepared by refluxing 2-[N′-tert-butoxycarbonyl-N′-(4-fluoro-phenyl)-hydrazino]-3-(4′,5′-difluoro-2′-methoxy-biphenyl-4-yloxymethyl)-benzoic acid methyl ester with hydrochloric acid in THF as described in the previous example. LRMS-ES(+) calcd for C₂₇H₁₉F₃N₂O₃ (M+H) 477.1, obsd 477.1; ¹H NMR (DMSO-d₆) δ (ppm) 10.72 (s, 1H), 7.95 (dd, J=9.2, 5.0 Hz, 2H), 7.75 (d, J=7.8 Hz, 1H), 7.71 (d, J=7.2 Hz, 1H), 7.44 (d, J=8.8 Hz, 2H), 7.30-7.43 (m, 3H), 7.19-7.28 (m, 2H), 7.11 (d, J=8.8 Hz, 2H), 5.34 (s, 2H), 3.76 (s, 3H).

### Example 12

### 7-(4′,5′-Difluoro-2′-methoxy-biphenyl-4-yloxymethyl)-2-pyridin-3-yl-1,2-dihydro-indazol-3-one

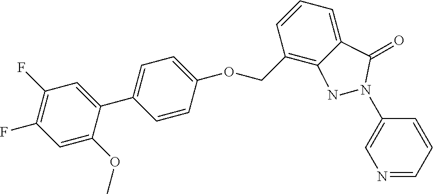

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-(N′-tert-butoxycarbonyl-N′-pyridin-3-yl-hydrazino)-3-(2′-methoxy-4′,5′-difluoro-biphenyl-4 yloxymethyl)-benzoic acid methyl ester was prepared from 2-bromo-3-(2′-methoxy-4′,5′-difluoro-biphenyl-4-yloxymethyl)-benzoic acid methyl ester and N-pyridin-3-yl-hydrazinecarboxylic acid tert-butyl ester. ¹H NMR (DMSO-d₆) δ (ppm) 9.39 (br. s., 1H), 8.83 (d, J=2.4 Hz, 1H), 8.30 (dd, J=4.7, 1.4 Hz, 1H), 7.90-8.02 (m, 1H), 7.72 (d, J=7.7 Hz, 1H), 7.65 (dd, J=7.7, 1.2 Hz, 1H), 7.35 (d, J=8.8 Hz, 2H), 7.31-7.39 (m, 2H), 7.23 (dd, J=13.1, 7.1 Hz, 1H), 7.04 (t, J=7.7 Hz, 1H), 6.79 (d, J=8.8 Hz, 2H), 4.91 (s, 2H), 3.75 (s, 3H), 3.74 (br. s., 3H), 1.24 (s, 9H).

7-(4′,5′-Difluoro-2′-methoxy-biphenyl-4-yloxymethyl)-2-pyridin-3-yl-1,2-dihydro-indazol-3-one was prepared by refluxing 2-(N′-tert-butoxycarbonyl-N′-pyridin-3-yl-hydrazino)-3-(4′,5′-difluoro-2′-methoxy-biphenyl-4-yloxymethyl)-benzoic acid methyl ester with hydrochloric acid in THF. LRMS-ES(+) calcd for C₂₆H₁₉F₂N₃O₃ (M+H) 460.1, obsd 460.0; ¹H NMR (DMSO-d₆) δ (ppm) 10.76 (s, 1H), 9.16 (d, J=2.4 Hz, 1H), 8.47 (dd, J=4.7, 1.4 Hz, 1H), 8.24-8.37 (m, 1H), 7.76 (t, J=8.0 Hz, 2H), 7.57 (dd, J=8.3, 4.7 Hz, 1H), 7.45 (d, J=8.8 Hz, 2H), 7.37 (dd, J=11.3, 9.5 Hz, 1H), 7.19-7.31 (m, 2H), 7.13 (d, J=8.8 Hz, 2H), 5.35 (s, 2H), 3.76 (s, 3H)

### Example 13

### 2-Pyridin-3-yl-7-(2′,4′,5′-trifluoro-biphenyl-4-yloxymethyl)-1,2-dihydro-indazol-3-one Hydrochloride

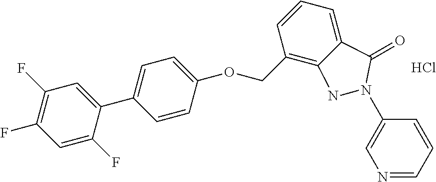

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-(N′-tert-butoxycarbonyl-N′-pyridin-3-yl-hydrazino)-3-(2′,4′,5′-trifluoro-biphenyl-4-yloxymethyl)-benzoic acid methyl ester was prepared from 2-bromo-3-(2′,4′,5′-trifluoro-biphenyl-4-yloxymethyl)-benzoic acid methyl ester and N-pyridin-3-yl-hydrazinecarboxylic acid tert-butyl ester. ¹H NMR (DMSO-d₆) δ (ppm) 9.41 (br. s., 1H), 8.82 (d, J=2.4 Hz, 1H), 8.29 (dd, J=4.5, 1.2 Hz, 1H), 7.93-7.99 (m, 1H), 7.73 (d, J=7.7 Hz, 1H), 7.57-7.70 (m, 3H), 7.44 (dd, J=8.7, 1.4 Hz, 2H), 7.33 (dd, J=8.5, 4.5 Hz, 1H), 7.04 (t, J=7.7 Hz, 1H), 6.86 (d, J=8.7 Hz, 2H), 4.93 (s, 2H), 3.74 (s, 3H), 1.24 (s, 9H).

2-Pyridin-3-yl-7-(2′,4′,5′-trifluoro-biphenyl-4-yloxymethyl)-1,2-dihydro-indazol-3-one was prepared by refluxing 2-(N′-tert-butoxycarbonyl-N′-pyridin-3-yl-hydrazino)-3-(2′,4′,5′-trifluoro-biphenyl-4-yloxymethyl)-benzoic acid methyl ester and hydrochloric acid in THF as described in the previous example. The reaction mixture was cooled to room temperature and the precipitate was filtered and washed with ether to give the desired compound as a hydrochloride salt. LRMS calcd for C₂₅H₁₆F₃N₃O₂ (M+H) 448.1, obsd 448.0; ¹H NMR (DMSO-d₆) δ (ppm) 11.07 (br. s., 1H), 9.33 (d, J=2.1 Hz, 1H), 8.56-8.64 (m, 2H), 7.75-7.87 (m, 3H), 7.59-7.73 (m, 2H), 7.54 (dd, J=8.8, 1.5 Hz, 2H), 7.29 (t, J=7.5 Hz, 1H), 7.21 (d, J=8.8 Hz, 2H), 5.41 (s, 2H).

### Example 14

### 7-(4′,5′-Difluoro-2′-chloro-biphenyl-4-yloxymethyl)-2-pyridin-3-yl-1,2-dihydro-indazol-3-one

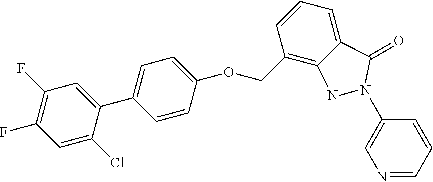

With a method similar to that used for the preparation of 2-(N′-tert-butoxycarbonyl-N′-methyl-hydrazino)-3-(4′,5′-difluoro-2′-methyl-biphenyl-4-yloxymethyl)-benzoic acid methyl ester, 2-(N′-tert-butoxycarbonyl-N′-pyridin-3-yl-hydrazino)-3-(2′-chloro-4′,5′-difluoro-biphenyl-4 yloxymethyl)-benzoic acid methyl ester was prepared from 2-bromo-3-(2′-chloro-4′,5′-difluoro-biphenyl-4-yloxymethyl)-benzoic acid methyl ester and N-pyridin-3-yl-hydrazinecarboxylic acid tert-butyl ester. ¹H NMR (DMSO-d₆) δ (ppm) 9.39 (br. s., 1H), 8.82 (d, J=2.4 Hz, 1H), 8.28 (dd, J=4.5, 1.2 Hz, 1H), 7.90-8.01 (m, 1H), 7.81 (dd, J=10.6, 7.5 Hz, 1H), 7.73 (d, J=7.2 Hz, 1H), 7.65 (dd, J=7.5, 1.2 Hz, 1H), 7.52 (dd, J=11.2, 8.8 Hz, 1H), 7.32 (d, J=8.8 Hz, 2H), 7.28-7.37 (m, 1H), 7.04 (t, J=7.5 Hz, 1H), 6.84 (d, J=8.8 Hz, 2H), 4.92 (s, 2H), 3.73 (s, 3H), 1.23 (s, 9H)

7-(4′,5′-Difluoro-2′-chloro-biphenyl-4-yloxymethyl)-2-pyridin-3-yl-1,2-dihydro-indazol-3-one was prepared by refluxing 2-(N′-tert-butoxycarbonyl-N′-pyridin-3-yl-hydrazino)-3-(4′,5′-difluoro-2′-chloro-biphenyl-4-yloxymethyl)-benzoic acid methyl ester with hydrochloric acid in THF as described in the previous example. LRMS-ES(+) calcd for C₂₅H₁₆ClF₂N₃O₂ (M+H) 464.1, obsd 464.0. ¹H-NMR (DMSO-d₆) δ (ppm) 11.10 (br, s, 1H), 9.33 (d, J=2.1 Hz, 1H), 8.58-8.66 (m, 2H), 7.76-7.89 (m, 4H), 7.56 (dd, J=11.3, 8.6 Hz, 1H), 7.42 (d, J=8.8 Hz, 2H), 7.30 (t, J=7.5 Hz, 1H), 7.20 (d, J=8.8 Hz, 2H), 5.41 (s, 2H).

### Example 15

### 2-((R)-2,3-Dihydroxy-propyl)-7-(2′,4′,5′-trifluoro-biphenyl-4-yloxymethyl)-1,2-dihydro-indazol-3-one

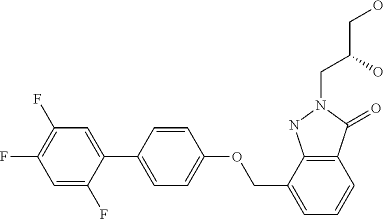

To a 15 mL thick glass tube was added 2-bromo-3-(2′,4′,5′-trifluoro-biphenyl-4-yloxymethyl)-benzoic acid methyl ester (902 mg, 2.0 mmol), N—((R)-2,2-dimethyl-[1,3]dioxolan-4-ylmethyl)-hydrazinecarboxylic acid tert-butyl ester (738 mg, 3.0 mmol), cesium carbonate (1.30 g, 4 mmol), and toluene (10 mL). The mixture was degassed with argon and palladium acetate (22.4 mg, 0.1 mmol), tri-tert-butylphosphonium tetrafluoroborate (29.0 mg, 0.1 mmol) were added. The sealed tube was stirred and heated at 120° C. for 5 hrs. The mixture was cooled down to room temperature and filtered. The solid was rinsed with ethyl acetate. The filtrate was concentrated and the residue was purified through a flash column chromatography (silica gel 50 g) eluted with ethyl acetate in hexanes (0% to 25% in 25 minutes) to provide 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester as a gummy material (658 mg, 54%). LRMS calcd for C₃₂H₃₅F₃N₂O₇ (m/e) calcd 616.24, obsd 617.1 (M+H, ES+); HRMS (ES+) calcd 639.2288 [M+Na]⁺, obsd 639.2291; ¹H-NMR (CDCl₃) δ (ppm) 9.60 (s, 1H), 7.94 (dd, J=8.0, 1.5 Hz, 1H), 7.66 (dd, J=7.8, 1.1 Hz, 1H), 7.40 (dd, J=8.7, 1.6 Hz, 2H), 7.15-7.26 (m, 1H), 6.99 (d, J=8.7 Hz, 2H), 6.97-7.04 (m, 1H), 6.95 (t, J=7.8 Hz, 1H), 5.08 (s, 2H), 4.41 (quin, J=5.9 Hz, 1H), 4.08 (dd, J=8.5, 6.2 Hz, 1H), 3.92 (s, 3H), 3.71 (dd, J=8.5, 5.8 Hz, 1H), 3.82 (br s, 2H), 1.44 (s, 3H), 1.37 (s, 3H), 1.30 (s, 9H).

To a 100 mL round bottom flask was added 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester (640 mg, 1.04 mmol), hydrochloric acid (3N, 10 mL), and THF (20 mL). The mixture was stirred and refluxed for 1.5 hr and then concentrated. The residue was extracted with ethyl acetate (80 mL) and water (30 mL). The organic layer was washed with brine (50 mL) and dried over sodium sulfate. The mixture was filtered and solvent was evaporated. The residue was treated with anhydrous ether (30 mL). The white crystalline solid was filtered to provide 2-((R)-2,3-dihydroxy-propyl)-7-(2′,4′,5′-trifluoro-biphenyl-4-yloxymethyl)-1,2-dihydro-indazol-3-one (361 mg, 78.3%). This material was analyzed by super critical chromatography with a chiral column (OD column) and compared with the corresponding racemate. The chiral purity was 100%. LRMS-ES(+) calcd for C₂₃H₁₉F₃N₂O₄ (m/e) 444.41, obsd 445.0 (M+H, ES+); HRMS (ES+) calcd 445.1370 [M+H]⁺, obsd 445.1370; ¹H-NMR (DMSO-d₆) δ (ppm) 10.22 (s, 1H), 7.58-7.71 (m, 4H), 7.52 (dd, J=8.7, 1.4 Hz, 2H), 7.17 (d, J=8.7 Hz, 2H), 7.10-7.20 (m, 1H), 5.29 (s, 2H), 5.11 (br s, 1H), 4.80 (br s, 1H), 3.76-3.98 (m, 3H), 3.27-3.46 (m, 2H).

### Example 16

### 2-((S)-2,3-Dihydroxy-propyl)-7-(2′,4′,5′-trifluoro-biphenyl-4-yloxymethyl)-1,2-dihydro-indazol-3-one

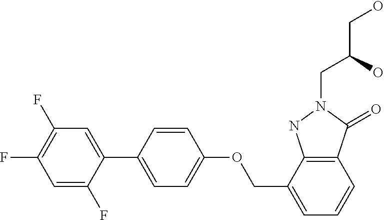

With the same method as described for the preparation of 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester, 2-[N′-tert-butoxycarbonyl-N′-((S)-2,2-dimethyl-[1,3]dioxolan-4-ylmethyl)-hydrazino]-3-(2′,4′,5′-trifluoro-biphenyl-4-yloxymethyl)-benzoic acid methyl ester was prepared from N—((S)-2,2-dimethyl-[1,3]dioxolan-4-ylmethyl)-hydrazinecarboxylic acid tert-butyl ester and 2-bromo-3-(2′,4′,5′-trifluoro-biphenyl-4-yloxymethyl)-benzoic acid methyl ester (86.4% yield). LRMS calcd for C₃₂H₃₅F₃N₂O₇ (m/e) calcd 616.24, obsd 615.1 (M−H, ES−); ¹H-NMR (CDCl₃) δ (ppm) 9.60 (s, 1H), 7.94 (dd, J=8.2, 1.5 Hz, 1H), 7.66 (d, J=7.5 Hz, 1H), 7.40 (dd, J=8.8, 1.5 Hz, 2H), 7.13-7.25 (m, 1H), 6.99 (d, J=8.8 Hz, 2H), 6.90-7.06 (m, 2H), 5.08 (s, 2H), 4.41 (dq, J=6.0, 5.8 Hz, 1H), 4.08 (dd, J=8.5, 6.0 Hz, 1H), 3.92 (s, 3H), 3.71 (dd, J=8.5, 6.0 Hz, 1H), 3.67 (br s, 2H), 1.44 (s, 3H), 1.37 (s, 3H), 1.30 (s, 9H).

2-((S)-2,3-Dihydroxy-propyl)-7-(2′,4′,5′-trifluoro-biphenyl-4-yloxymethyl)-1,2-dihydro-indazol-3-one was prepared by refluxing 2-[N′-tert-butoxycarbonyl-N′-((S)-2,2-dimethyl-[1,3]dioxolan-4-ylmethyl)-hydrazino]-3-(2′,4′,5′-trifluoro-biphenyl-4-yloxymethyl)-benzoic acid methyl ester with hydrochloric acid in THF as described for the preparation of 2-((R)-2,3-dihydroxy-propyl)-7-(2′,4′,5′-trifluoro-biphenyl-4-yloxymethyl)-1,2-dihydro-indazol-3-one (73.3% yield). This material was analyzed by super critical chromatography with a chiral column (OD column) and compared with the corresponding racemate. The chiral purity was 100%. LRMS calcd for C₂₃H₁₉F₃N₂O₄ (m/e) calcd 444.13, obsd 445.0 (M+H, ES+); ¹H-NMR (DMSO-d₆) δ (ppm) 10.22 (br s, 1H), 7.57-7.72 (m, 4H), 7.52 (dd, J=8.8, 1.4 Hz, 2H), 7.17 (d, J=8.8 Hz, 2H), 7.08-7.22 (m, 1H), 5.30 (s, 2H), 5.10 (br s, 1H), 4.81 (br s, 1H), 3.74-3.98 (m, 3H), 3.28-3.48 (m, 2H).

### Example 17

### 7-(4′,5′-Difluoro-2′-methoxy-biphenyl-4-yloxymethyl)-2-((R)-2,3-dihydroxy-propyl)-1,2-dihydro-indazol-3-one

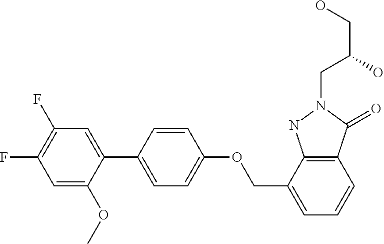

With the same method as described for the preparation of 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester, 2-[N′-tert-butoxycarbonyl-N′-((R)-2,2-dimethyl-[1,3]dioxolan-4-ylmethyl)-hydrazino]-3-(4′,5′-difluoro-2′-methoxy-biphenyl-4-yloxymethyl)-benzoic acid methyl ester was prepared from N#R)-2,2-dimethyl-[1,3]dioxolan-4-ylmethyl)-hydrazinecarboxylic acid tert-butyl ester and 2-bromo-3-(4′,5′-difluoro-2′-methoxy-biphenyl-4-yloxymethyl)-benzoic acid methyl (97% yield). LRMS calcd for C₃₃H₃₈F₂N₂O₈ (m/e) calcd 628.26, obsd 629.3 (M+H, ES+); ¹H-NMR (CDCl₃) δ (ppm) 9.59 (s, 1H), 7.93 (dd, J=7.9, 1.2 Hz, 1H), 7.68 (dd, J=7.8, 1.2 Hz, 1H), 7.38 (d, J=8.9 Hz, 2H), 7.11 (dd, J=10.9, 9.1 Hz, 1H), 6.96 (d, J=8.9 Hz, 2H), 6.92-6.98 (m, 1H), 6.78 (dd, J=12.2, 6.7 Hz, 1H), 5.07 (s, 2H), 4.42 (quin, J=6.0 Hz, 1H), 4.08 (dd, J=8.5, 6.0 Hz, 1H), 3.92 (s, 3H), 3.77 (s, 3H), 3.72 (dd, J=8.5, 6.0 Hz, 1H), 3.54 (br s, 2H), 1.44 (s, 3H), 1.37 (s, 3H), 1.30 (s, 9H).

7-(4′,5′-Difluoro-2′-methoxy-biphenyl-4-yloxymethyl)-2-((R)-2,3-dihydroxy-propyl)-1,2-dihydro-indazol-3-one was prepared by refluxing 2-[N′-tert-butoxycarbonyl-N′-((R)-2,2-dimethyl-[1,3]dioxolan-4-ylmethyl)-hydrazino]-3-(4′,5′-difluoro-2′-methoxy-biphenyl-4-yloxymethyl)-benzoic acid methyl ester with hydrochloric acid in THF as described for the preparation of 2-((R)-2,3-dihydroxy-propyl)-7-(2′,4′,5′-trifluoro-biphenyl-4-yloxymethyl)-1,2-dihydro-indazol-3-one (71% yield). LRMS calcd for C₂₄H₂₂F₂N₂O₅ (m/e) calcd 456.15, obsd 457.1 (M+H, ES+); HRMS (ES+) calcd 457.1570 [M+H]⁺, obsd 457.1569; ¹H-NMR (DMSO-d₆) δ (ppm) 10.21 (s, 1H), 7.64 (dd, J=7.5, 0.9 Hz, 1H), 7.60 (dd, J=7.5, 0.9 Hz, 1H), 7.43 (d, J=8.9 Hz, 2H), 7.36 (dd, J=11.5, 9.4 Hz, 1H), 7.24 (dd, J=13.1, 7.1 Hz, 1H), 7.14 (t, J=7.5 Hz, 1H), 7.09 (d, J=8.9 Hz, 2H), 5.27 (s, 2H), 5.07 (br s, 1H), 4.82 (br s, 1H), 3.78-4.01 (m, 3H), 3.76 (s, 3H), 3.29-3.46 (m, 2H).

### Example 18

### 7-(4′,5′-Difluoro-2′-methoxy-biphenyl-4-yloxymethyl)-2-((S)-2,3-dihydroxy-propyl)-1,2-dihydro-indazol-3-one

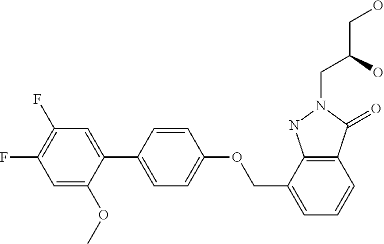

With the same method as described for the preparation of 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester, 2-[N′-tert-butoxycarbonyl-N′-((S)-2,2-dimethyl-[1,3]dioxolan-4-ylmethyl)-hydrazino]-3-(4′,5′-difluoro-2′-methoxy-biphenyl-4-yloxymethyl)-benzoic acid methyl ester was prepared from N4S)-2,2-dimethyl-[1,3]dioxolan-4-ylmethyl)-hydrazinecarboxylic acid tert-butyl ester and 2-bromo-3-(4′,5′-difluoro-2′-methoxy-biphenyl-4-yloxymethyl)-benzoic acid methyl (93% yield). LRMS calcd for C₃₃H₃₈F₂N₂O₈ (m/e) calcd 628.26, obsd 629.3 (M+H, ES+).

7-(4′,5′-Difluoro-2′-methoxy-biphenyl-4-yloxymethyl)-2-(S)-2,3-dihydroxy-propyl)-1,2-dihydro-indazol-3-one was prepared by refluxing 2-[N′-tert-butoxycarbonyl-N′-((S)-2,2-dimethyl-[1,3]dioxolan-4-ylmethyl)-hydrazino]-3-(4′,5′-difluoro-2′-methoxy-biphenyl-4-yloxymethyl)-benzoic acid methyl ester with hydrochloric acid in THF as described for the preparation of 2-((R)-2,3-dihydroxy-propyl)-7-(2′,4′,5′-trifluoro-biphenyl-4-yloxymethyl)-1,2-dihydro-indazol-3-one (71% yield). LRMS calcd for C₂₄H₂₂F₂N₂O₅ (m/e) calcd 456.15, obsd 457.1 (M+H, ES+); HRMS (ES+) calcd 457.1570 [M+H]⁺, obsd 457.1569; ¹H-NMR (DMSO-d₆) δ (ppm) 10.21 (s, 1H), 7.64 (d, J=7.5 Hz, 1H), 7.60 (d, J=7.5 Hz, 1H), 7.43 (d, J=8.8 Hz, 2H), 7.36 (dd, J=11.5, 9.4 Hz, 1H), 7.24 (dd, J=13.0, 6.9 Hz, 1H), 7.14 (t, J=7.5 Hz, 1H), 7.09 (d, J=8.8 Hz, 2H), 5.27 (s, 2H), 4.98 (br s, 2H), 3.77-4.00 (m, 3H), 3.76 (s, 3H), 3.28-3.47 (m, 2H).

### Example 19

### 7-(4′,5′-Difluoro-2′-methyl-biphenyl-4-yloxymethyl)-2-((R)-2,3-dihydroxy-propyl)-1,2-dihydro-indazol-3-one

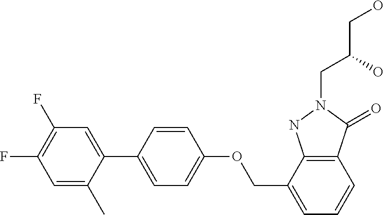

With the same method as described for the preparation of 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester, 2-[N′-tert-butoxycarbonyl-N′-((R)-2,2-dimethyl-[1,3]dioxolan-4-ylmethyl)-hydrazino]-3-(4′,5′-difluoro-2′-methyl-biphenyl-4-yloxymethyl)-benzoic acid methyl ester was prepared from N—((R)-2,2-dimethyl-[1,3]dioxolan-4-ylmethyl)-hydrazinecarboxylic acid tert-butyl ester and 2-bromo-3-(4′,5′-difluoro-2′-methyl-biphenyl-4-yloxymethyl)-benzoic acid methyl (73% yield). LRMS calcd for C₃₃H₃₈F₂N₂O₇ (m/e) calcd 612.26, obsd 611.2 (M−H, ES−). HRMS (ES+) calcd 635.2539 [M+Na]⁺, obsd 635.2541; ¹H-NMR (CDCl₃) δ (ppm) 9.59 (s, 1H), 7.94 (dd, J=7.9 Hz, 1.4 Hz, 1H), 7.69 (dd, J=7.7, 1.4 Hz, 1H), 7.17 (d, J=8.9 Hz, 2H), 6.96 (d, J=8.9 Hz, 2H), 6.94-7.07 (m, 3H), 5.08 (s, 2H), 4.41 (dt, J=11.7, 5.8 Hz, 1H), 4.08 (dd, J=8.5, 6.2 Hz, 1H), 3.92 (s, 3H), 3.82 (br s, 2H), 3.72 (dd, J=8.5, 5.8 Hz, 1H), 2.20 (s, 3H), 1.44 (s, 3H), 1.37 (s, 3H), 1.30 (s, 9H).

7-(4′,5′-Difluoro-2′-methyl-biphenyl-4-yloxymethyl)-2-((R)-2,3-dihydroxy-propyl)-1,2-dihydro-indazol-3-one was prepared by refluxing 2-[N′-tert-butoxycarbonyl-N′-((R)-2,2-dimethyl-[1,3]dioxolan-4-ylmethyl)-hydrazino]-3-(4′,5′-difluoro-2′-methyl-biphenyl-4-yloxymethyl)-benzoic acid methyl ester with hydrochloric acid in THF as described for the preparation of 2-((R)-2,3-dihydroxy-propyl)-7-(2′,4′,5′-trifluoro-biphenyl-4-yloxymethyl)-1,2-dihydro-indazol-3-one (71% yield). LRMS calcd for C₂₄H₂₂F₂N₂O₄ (m/e) calcd 440.15, obsd 441.1 (M+H, ES+); HRMS (ES+) calcd 441.1621 [M+H]⁺, obsd 441.1620; ¹H-NMR (DMSO-d₆) δ (ppm) 10.23 (s, 1H), 7.65 (d, J=7.8 Hz, 1H), 7.62 (d, J=7.5 Hz, 1H), 7.38 (dd, J=11.8, 8.5 Hz, 1H), 7.31 (d, J=8.8 Hz, 2H), 7.23 (dd, J=11.5, 8.5 Hz, 1H), 7.12 (d, J=8.8 Hz, 2H), 7.08-7.17 (m, 1H), 5.28 (s, 2H), 5.03 (br s, 2H), 3.75-4.01 (m, 3H), 3.30-3.47 (m, 2H), 2.20 (s, 3H).

### Example 20

### 7-(4′,5′-Difluoro-2′-methyl-biphenyl-4-yloxymethyl)-2-((S)-2,3-dihydroxy-propyl)-1,2-dihydro-indazol-3-one

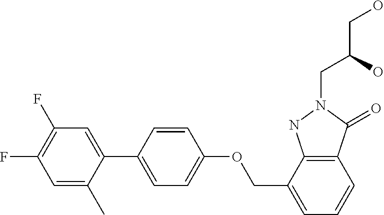

With the same method as described for the preparation of 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester, 2-[N′-tert-butoxycarbonyl-N′-((S)-2,2-dimethyl-[1,3]dioxolan-4-ylmethyl)-hydrazino]-3-(4′,5′-difluoro-2′-methyl-biphenyl-4-yloxymethyl)-benzoic acid methyl ester was prepared from N4S)-2,2-dimethyl-[1,3]dioxolan-4-ylmethyl)-hydrazinecarboxylic acid tert-butyl ester and 2-bromo-3-(4′,5′-difluoro-2′-methyl-biphenyl-4-yloxymethyl)-benzoic acid methyl (72% yield). LRMS calcd for C₃₃H₃₈F₂N₂O₇ (m/e) calcd 612.26, obsd 611.1 (M−H, ES−). HRMS (ES+) calcd 635.2539 [M+Na]⁺, obsd 635.2537; ¹H-NMR (DMSO-d₆) δ (ppm) 9.07 (br s, 1H), 7.75 (d, J=7.5 Hz, 1H), 7.64 (dd, J=7.5, 1.5 Hz, 1H), 7.37 (dd, J=12.1, 8.2 Hz, 1H), 7.29 (d, J=8.8 Hz, 2H), 7.23 (dd, J=11.6, 8.3 Hz, 1H), 7.03 (d, J=8.8 Hz, 2H), 6.98 (t, J=7.5 Hz, 1H), 5.05 (s, 2H), 4.31 (quin, J=5.9 Hz, 1H), 4.01 (dd, J=8.5, 6.3 Hz, 1H), 3.84 (s, 3H), 3.65 (dd, J=8.5, 5.4 Hz, 1H), 3.53 (br s, 2H), 2.19 (s, 3H), 1.32 (s, 3H), 1.26 (s, 3H), 1.21 (s, 9H).

7-(4′,5′-Difluoro-2′-methyl-biphenyl-4-yloxymethyl)-2-((S)-2,3-dihydroxy-propyl)-1,2-dihydro-indazol-3-one was prepared by refluxing 2-[N′-tert-butoxycarbonyl-N′-((S)-2,2-dimethyl-[1,3]dioxolan-4-ylmethyl)-hydrazino]-3-(4′,5′-difluoro-2′-methyl-biphenyl-4-yloxymethyl)-benzoic acid methyl ester with hydrochloric acid in THF as described for the preparation of 2-((R)-2,3-dihydroxy-propyl)-7-(2′,4′,5′-trifluoro-biphenyl-4-yloxymethyl)-1,2-dihydro-indazol-3-one (80% yield). LRMS calcd for C₂₄H₂₂F₂N₂O₄ (m/e) calcd 440.15, obsd 441.1 (M+H, ES+); HRMS (ES+) calcd 441.1621 [M+H]⁺, obsd 441.1619; ¹H-NMR (DMSO-d₆) δ (ppm) 10.23 (s, 1H), 7.65 (d, J=7.8 Hz, 1H), 7.62 (d, J=7.2 Hz, 1H), 7.38 (dd, J=11.9, 8.6 Hz, 1H), 7.31 (d, J=8.8 Hz, 2H), 7.24 (dd, J=11.5, 8.5 Hz, 1H), 7.12 (d, J=8.8 Hz, 2H), 7.10-7.18 (m, 1H), 5.28 (s, 2H), 5.12 (br s, 1H), 4.83 (br s, 1H), 3.72-4.03 (m, 3H), 3.26-3.48 (m, 2H), 2.20 (s, 3H).

### Example 21

### 7-(2′-Chloro-4′,5′-difluoro-biphenyl-4-yloxymethyl)-2-((R)-2,3-dihydroxy-propyl)-1,2-dihydro-indazol-3-one

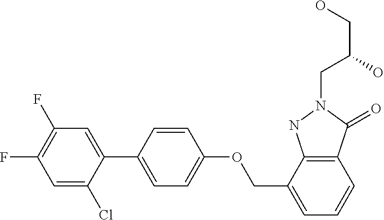

With the same method as described for the preparation of 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester, 2-N′-tert-butoxycarbonyl-N′-((R)-2,2-dimethyl-[1,3]dioxolan-4-ylmethyl)-hydrazino]-3-(2′-chloro-4′,5′-difluoro-biphenyl-4-yloxymethyl)-benzoic acid methyl ester was prepared from 2-bromo-3-(2′-chloro-4′,5′-difluoro-biphenyl-4-yloxymethyl)-benzoic acid methyl ester and N—((R)-2,2-dimethyl-[1,3]dioxolan-4-ylmethyl)-hydrazinecarboxylic acid tert-butyl ester (60% yield). LRMS calcd for C₃₂H₃₅ClF₂N₂O₇ (m/e) calcd 632.21, obsd 633.1 (M+H, ES+). ¹H-NMR (CDCl₃) δ (ppm) 9.59 (br s, 1H), 7.94 (d, J=7.8 Hz, 1H), 7.67 (d, J=7.2 Hz, 1H), 7.21-7.36 (m, 3H), 7.15 (dd, J=10.6, 8.5 Hz, 1H), 6.87-7.03 (m, 3H), 5.08 (s, 2H), 4.41 (t, J=5.6 Hz, 1H), 4.07 (t, J=7.4 Hz, 1H), 3.91 (s, 3H), 3.71 (dd, J=8.2, 6.0 Hz, 1H), 3.61 (br s, 2H), 1.43 (s, 3H), 1.36 (s, 3H), 1.29 (s, 9H).

7-(2′-Chloro-4′,5′-difluoro-biphenyl-4-yloxymethyl)-2-((R)-2,3-dihydroxy-propyl)-1,2-dihydro-indazol-3-one was prepared by refluxing 2-N′-tert-butoxycarbonyl-N′-((R)-2,2-dimethyl-[1,3]dioxolan-4-ylmethyl)-hydrazino]-3-(2′-chloro-4′,5′-difluoro-biphenyl-4-yloxymethyl)-benzoic acid methyl ester with hydrochloric acid in THF as described for the preparation of 2-((R)-2,3-dihydroxy-propyl)-7-(2′,4′,5′-trifluoro-biphenyl-4-yloxymethyl)-1,2-dihydro-indazol-3-one (57.4% yield). LRMS calcd for C₂₃H₁₉ClF₂N₂O₄ (m/e) calcd 460.10, obsd 461.0 (M+H, ES+); ¹H-NMR (DMSO-d₆) δ (ppm) 10.24 (s, 1H), 7.82 (dd, J=10.4, 7.7 Hz, 1H), 7.65 (d, J=7.8 Hz, 1H), 7.62 (d, J=7.5 Hz, 1H), 7.55 (dd, J=11.2, 8.8 Hz, 1H), 7.40 (d, J=8.8 Hz, 2H), 7.15 (d, J=8.8 Hz, 2H), 7.10-7.17 (m, 1H), 5.29 (s, 2H), 5.10 (br s, 1H), 4.79 (br s, 1H), 3.74-4.00 (m, 3H), 3.28-3.45 (m, 2H).

### Example 22

### 7-(2′-Chloro-4′,5′-difluoro-biphenyl-4-yloxymethyl)-2-((S)-2,3-dihydroxy-propyl)-1,2-dihydro-indazol-3-one

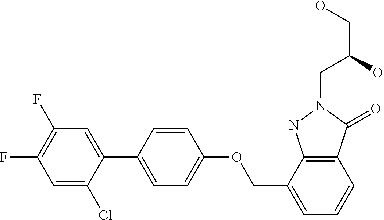

With the same method as described for the preparation of 2-[N′-tert-butoxycarbonyl-N′-((R)-2,2-dimethyl-[1,3]dioxolan-4-ylmethyl)-hydrazino]-3-(2′,4′,5′-trifluoro-biphenyl-4-yloxymethyl)-benzoic acid methyl ester, 2-N′-tert-butoxycarbonyl-N′-((S)-2,2-dimethyl-[1,3]dioxolan-4-ylmethyl)-hydrazino]-3-(2′-chloro-4′,5′-difluoro-biphenyl-4-yloxymethyl)-benzoic acid methyl ester was prepared from 2-bromo-3-(2′-chloro-4′,5′-difluoro-biphenyl-4-yloxymethyl)-benzoic acid methyl ester and N—((S)-2,2-dimethyl-[1,3]dioxolan-4-ylmethyl)-hydrazinecarboxylic acid tert-butyl ester (84% yield). LRMS calcd for C₃₂H₃₅ClF₂N₂O₇ (m/e) calcd 632.21, obsd 655.1 (M+Na, ES+). ¹H-NMR (CDCl₃) δ (ppm) 9.58 (br s, 1H), 7.93 (dd, J=7.8, 1.5 Hz, 1H), 7.66 (dd, J=7.5, 0.9 Hz, 1H), 7.27-7.34 (m, 3H), 7.14 (dd, J=10.7, 8.3 Hz, 1H), 6.90-7.01 (m, 3H), 5.07 (s, 2H), 4.40 (quin, J=5.9 Hz, 1H), 4.07 (dd, J=8.5, 6.0 Hz, 1H), 3.91 (s, 3H), 3.70 (dd, J=8.5, 6.0 Hz, 1H), 3.66 (br s, 2H), 1.43 (s, 3H), 1.36 (s, 3H), 1.29 (s, 9H).

7-(2′-Chloro-4′,5′-difluoro-biphenyl-4-yloxymethyl)-2-((S)-2,3-dihydroxy-propyl)-1,2-dihydro-indazol-3-one was prepared by refluxing 2-N′-tert-butoxycarbonyl-N′-((S)-2,2-dimethyl-[1,3]dioxolan-4-ylmethyl)-hydrazino]-3-(2′-chloro-4′,5′-difluoro-biphenyl-4-yloxymethyl)-benzoic acid methyl ester with hydrochloric acid in THF as described for the preparation of 2-((R)-2,3-dihydroxy-propyl)-7-(2′,4′,5′-trifluoro-biphenyl-4-yloxymethyl)-1,2-dihydro-indazol-3-one (82% yield). LRMS calcd for C₂₃H₁₉ClF₂N₂O₄ (m/e) calcd 460.10, obsd 461.0 (M+H, ES+); ¹H-NMR (DMSO-d₆) δ (ppm) 10.24 (br s, 1H), 7.82 (dd, J=10.3, 7.8 Hz, 1H), 7.59-7.71 (m, 2H), 7.55 (dd, J=11.0, 8.9 Hz, 1H), 7.40 (d, J=8.5 Hz, 2H), 7.09-7.20 (m, 3H), 5.29 (s, 2H), 5.11 (br s, 1H), 4.80 (br s, 1H), 3.72-4.00 (m, 3H), 3.28 (br s, 2H).

### Example 23

### Glycogen Synthase (GS) Assay

The following tests were carried out in order to determine the activity of the compounds of formula (I).

Twelve μL per well of substrate solution containing glycogen (4.32 mg/ml), 2.67 mM UDP-glucose, 21.6 mM phospho(enol)pyruvate and 2.7 mM NADH in 30 mM glycylglycine, pH 7.3 buffer was added into a polystyrene 384-well assay plate (BD Biosciences).

Compound solutions (8 μL/well) at various concentrations (0-300 μM) were added to the assay plate (columns 5-24). Compound solution contains 30 mM glycylglycine, pH 7.3, 40 mM KCl, 20 mM MgCl₂, 9.2% DMSO, with (columns 15-24) or without (columns 5-14) 20 mM glucose 6-phosphate.

Enzyme solution (12 μL/well) containing glycogen synthase (16.88 μg/ml), pyruvate kinase (0.27 mg/ml), lactate dehydrogenase (0.27 mg/ml) in 50 mM Tris-HCl, pH 8.0, 27 mM DTT and bovine serum albumin (BSA, 0.2 mg/ml) was added to the assay plate (columns 3-24). As a blank control, enzyme solution without glycogen synthase was added into the top half wells of columns 1-2. To the bottom half wells of columns 1-2 were added a known activator, glucose 6-phosphate (at final concentration 5 mM) in addition to the enzyme solution. The reaction mixture was incubated at room temperature. The assay plate was then read for absorbance at 340 nm on an Envision reader every 3 minutes up to a total of 15 minutes.

The enzyme activity (with or without compound) was calculated by the reaction rate and represented by the optical density change (δOD) per minute. Percent stimulation of glycogen synthase activity by a compound at various concentrations was calculated by the following formula:

% stimulation=100*Rs/Rt,

wherein Rs is the reaction rate of the enzyme in the presence of compound and Rt is the reaction rate of the enzyme in the absence of compound.

SC₂₀₀ is defined as the compound concentration that is needed to stimulate 200% of the enzyme activity. EC₅₀ is defined as the compound concentration that is needed to give 50% maximum activation.

Compounds from Example 1 through Example 22 were assayed according to assay procedures described above and the result is listed in Table 1.

TABLE 1
Glycogen Synthase Activation Potency of Exampled Compounds

| Example Number | GS SC₂₀₀ (μM) | GS EC₅₀ (μM) |
|---|---|---|
| 1 | 0.13 | 0.24 |
| 2 | 0.49 | 1.78 |
| 3 | 0.04 | 0.33 |
| 4 | 0.69 | 1.20 |
| 5 | 0.06 | 0.20 |
| 6 | 0.52 | 0.79 |
| 7 | 0.11 | 0.37 |
| 8 | 0.22 | 0.77 |
| 9 | 0.33 | 0.42 |
| 10 | 1.86 | 1.47 |
| 11 | 2.01 | 1.37 |
| 12 | 0.60 | 0.78 |
| 13 | 2.75 | 4.01 |
| 14 | 1.14 | 1.86 |
| 15 | 0.32 | 0.68 |
| 16 | 0.41 | 0.58 |
| 17 | 0.06 | 0.27 |
| 18 | 0.07 | 0.25 |
| 19 | 0.10 | 0.23 |
| 20 | 0.11 | 0.34 |
| 21 | 0.25 | 0.26 |
| 22 | 0.16 | 0.30 |

It is to be understood that the invention is not limited to the particular embodiments of the invention described above, as variations of the particular embodiments may be made and still fall within the scope of the appended claims.

What is claimed is:

1. A compound of Formula (I): 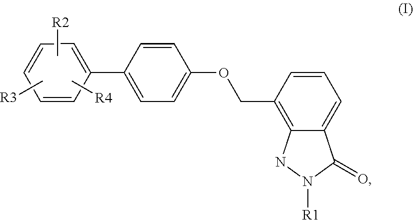
wherein:
R1 is heteroaryl, unsubstituted aryl, aryl substituted with halogen, unsubstituted lower alkyl or lower alkyl mono- or bi-substituted with hydroxy, alkoxy or —COOH; and
R2, R3, R4, independently of each other, is halogen, lower alkyl or alkoxy,
or a pharmaceutically acceptable salt thereof.

2. The compound according to claim 1, wherein R1 is lower alkyl; and R2, R3, R4, independently of each other, is halogen, lower alkyl or alkoxy.

3. The compound according to claim 1, wherein R1 is lower alkyl mono- or bi-substituted with hydroxy, alkoxy or —COOH; and R2, R3, R4, independently of each other, is halogen, lower alkyl or alkoxy.

4. The compound according to claim 1, wherein R1 is lower alkyl; R2 and R3 are halogen; and R4 is lower alkyl.

5. The compound according to claim 1, wherein R2 and R3 are halogen and R4 is lower alkyl.

6. The compound according to claim 1, wherein R1 is methyl, acetic acid, methoxy-ethyl, hydroxy-ethyl or dihydroxy-propyl.

7. The compound according to claim 1, wherein R1 is phenyl, fluoro-phenyl or pyridinyl.

8. The compound according to claim 1, wherein R2 is fluorine or chlorine.

9. The compound according to claim 1, wherein R3 is fluorine or chlorine.

10. The compound according to claim 1, wherein R4 is chlorine, fluorine, methyl or methoxy.

11. The compound according to claim 1, wherein said compound is:
7-(4′,5′-Difluoro-2′-methyl-biphenyl-4-yloxymethyl)-2-methyl-1,2-dihydro-indazol-3-one;
2-Methyl-7-(2′,4′,5′-trifluoro-biphenyl-4-yloxymethyl)-1,2-dihydro-indazol-3-one;
7-(4′,5′-Difluoro-2′-methoxy-biphenyl-4-yloxymethyl)-2-methyl-1,2-dihydro-indazol-3-one;
[3-oxo-7-(2′,4′,5′-trifluoro-biphenyl-4-yloxymethyl)-1,3-dihydro-indazol-2-yl]-acetic acid;
[7-(4′,5′-Difluoro-2′-methoxy-biphenyl-4-yloxymethyl)-3-oxo-1,3-dihydro-indazol-2-yl]-acetic acid;
2-(2-Methoxy-ethyl)-7-(2′,4′,5′-trifluoro-biphenyl-4-yloxymethyl)-1,2-dihydro-indazol-3-one Hydrochloride;
7-(4′,5′-Difluoro-2′-methoxy-biphenyl-4-yloxymethyl)-2-(2-methoxy-ethyl)-1,2-dihydro-indazol-3-one;
2-(2-Hydroxy-ethyl)-7-(2′,4′,5′-trifluoro-biphenyl-4-yloxymethyl)-1,2-dihydro-indazol-3-one;
[7-(4′,5′-Difluoro-2′-methyl-biphenyl-4-yloxymethyl)-3-oxo-1,3-dihydro-indazol-2-yl]-acetic acid hydrochloride;
7-(4′,5′-Difluoro-2′-methyl-biphenyl-4-yloxymethyl)-2-(4-fluoro-phenyl)-1,2-dihydro-indazol-3-one;
7-(4′,5′-Difluoro-2′-methoxy-biphenyl-4-yloxymethyl)-2-(4-fluoro-phenyl)-1,2-dihydro-indazol-3-one;
7-(4′,5′-Difluoro-2′-methoxy-biphenyl-4-yloxymethyl)-2-pyridin-3-yl-1,2-dihydro-indazol-3-one;
2-Pyridin-3-yl-7-(2′,4′,5′-trifluoro-biphenyl-4-yloxymethyl)-1,2-dihydro-indazol-3-one Hydrochloride;
7-(4′,5′-Difluoro-2′-chloro-biphenyl-4-yloxymethyl)-2-pyridin-3-yl-1,2-dihydro-indazol-3-one;
2-((R)-2,3-Dihydroxy-propyl)-7-(2′,4′,5′-trifluoro-biphenyl-4-yloxymethyl)-1,2-dihydro-indazol-3-one;
2-((S)-2,3-Dihydroxy-propyl)-7-(2′,4′,5′-trifluoro-biphenyl-4-yloxymethyl)-1,2-dihydro-indazol-3-one;
7-(4′,5′-Difluoro-2′-methoxy-biphenyl-4-yloxymethyl)-2-((R)-2,3-dihydroxy-propyl)-1,2-dihydro-indazol-3-one;
7-(4′,5′)-Difluoro-2′-methoxy-biphenyl-4-yloxymethyl)-2-((S)-2,3-dihydroxy-propyl)-1,2-dihydro-indazol-3-one;
7-(4′,5′)-Difluoro-2′-methyl-biphenyl-4-yloxymethyl)-2-((R)-2,3-dihydroxy-propyl)-1,2-dihydro-indazol-3-one;
7-(4′,5′)-Difluoro-2′-methyl-biphenyl-4-yloxymethyl)-2-((S)-2,3-dihydroxy-propyl)-1,2-dihydro-indazol-3-one;
7-(2′-Chloro-4′,5′-difluoro-biphenyl-4-yloxymethyl)-2-((R)-2,3-dihydroxy-propyl)-1,2-dihydro-indazol-3-one; and
7-(2′-Chloro-4′,5′-difluoro-biphenyl-4-yloxymethyl)-2-((S)-2,3-dihydroxy-propyl)-1,2-dihydro-indazol-3-one.

12. A pharmaceutical composition, comprising a therapeutically effective amount of a compound according to claim 1, or a pharmaceutically acceptable salt thereof, and a pharmaceutically acceptable carrier and/or adjuvant.
